SEQUENCE LISTING (1 sequence)

SEQ ID NO: 1 - DNA, 19 bp, synthetic construct
  source 1..19: mol_type = other DNA
atcgtagtaagtaccaatg

(19) United States
(12) Patent Application Publication
(10) Pub. No.: US 2026/0076971 A1
(43) Pub. Date: Mar. 19, 2026

(54) METHODS OF TREATING EPILEPSY AND REDUCING THE INCIDENCE OF SEIZURES

(71) Applicant: Wisconsin Alumni Research Foundation, Madison, WI (US)

(72) Inventors: Avtar Singh Roopra, Madison, WI (US); Olivia Rose Hoffman, Madison, WI (US)

(73) Assignee: Wisconsin Alumni Research Foundation, Madison, WI (US)

(21) Appl. No.: 19/399,203

(22) Filed: Nov. 24, 2025

Related U.S. Application Data
(63) Continuation-in-part of application No. 19/223,540, filed on May 30, 2025.
(60) Provisional application No. 63/654,280, filed on May 31, 2024.
(60) Provisional application No. 63/903,349, filed on Oct. 22, 2025.

(51) Int. Cl. A61K 31/519; A61K 9/00; A61K 45/06; A61P 25/14

(52) U.S. Cl. CPC A61K 31/519; A61K 9/0053; A61K 45/06; A61P 25/14

(57) ABSTRACT

In an aspect, a method of treating a patient with epilepsy or suspected of having epilepsy includes administering a Janus Kinase 1 or 1/3 inhibitor to the patient. The Janus Kinase 1 or 1/3 inhibitor can be administered for a first period of time, and then subsequently withdrawn for a second period of time, wherein seizures are suppressed in the patient during the second period of time.

### CROSS-REFERENCE TO RELATED APPLICATIONS

This application is a continuation-in-part of U.S. application Ser. No. 19/223,540, filed May 30, 2025, which claims priority to U.S. Provisional Application 63/654,280 filed on May 31, 2024, and also claims priority to U.S. Provisional Application 63/903,349, filed Oct. 22, 2025, which are incorporated herein by reference in their entirety.

### STATEMENT REGARDING FEDERALLY SPONSORED RESEARCH & DEVELOPMENT

This invention was made with government support under NS108756 awarded by the National Institutes of Health. The government has certain rights in the invention.

### SEQUENCE LISTING

The Instant Application contains a Sequence Listing which has been submitted electronically in XML format and is hereby incorporated by reference in its entirety. Said XML copy, created on May 5, 2025, is named “SEQ_LIST_107668322_P240298US02.xml” and is 1,958 bytes in size. The Sequence Listing does not go beyond the disclosure in the application as filed.

### FIELD OF THE DISCLOSURE

The present disclosure is related to methods of treating epilepsy and reducing the occurrence of seizures.

### BACKGROUND

Epilepsy is the fourth most prevalent neurological disorder with over 50 million cases worldwide. Arising from a myriad of etiologies including gene mutations and environmental insults, the unifying characteristic of the epilepsies is the emergence of spontaneous, recurrent seizures (SRS). Anticonvulsants aimed at controlling seizures are the current first-line treatments for epilepsy. No current treatments have been shown to prevent, let alone reverse, disease progression, and patients often remain on medication for life, as no treatments are disease modifying. Further, despite the introduction of 12 new antiseizure drugs in the last 20 years, 30% of patients are drug resistant, a rate that has remained constant since 1850. Finally, with few exceptions, long-term use of many anti-epileptic drugs is associated with a marked decline in cognition, highlighting the need for transient treatments with enduring effects that address cognitive comorbidities, not just seizures.

In acquired epilepsies, epileptogenesis is the process that links the eventual propagation of spontaneous seizures to an acute, initiating traumatic insult. Status epilepticus (SE), a severe bout of unremitting seizures, is one of the most common methods for modeling epileptogenesis. This process is associated with a number of changes in the brain including molecular, cellular, and network alterations in plasticity and inflammation. Many signaling cascades exhibit a transient period of explosive activation that abates within days. How these cascades react to the onset of spontaneous seizures in chronic epilepsy is poorly characterized, and it is not known whether mechanisms invoked early after insult are reengaged upon disease establishment.

Antiepileptogenic therapies aim to prevent progression to spontaneous seizures through early intervention, although the lack of biomarkers for epilepsy makes this approach challenging in clinical practice. Disease modifying therapy would aim to slow or reverse the progression of chronic epilepsy after spontaneous seizures are manifested. Currently, there are no disease modifying therapeutic interventions that retard disease in an enduring manner upon brief administration.

What is needed are new therapies for the treatment of epilepsy, particularly therapies that can reduce the incidence of seizures.

### BRIEF SUMMARY

In an aspect, a method of treating a patient with epilepsy or suspected of having epilepsy comprises administering a Janus Kinase inhibitor such as a Janus Kinase 1 or 1/3 inhibitor to the patient. The Janus Kinase inhibitor can be administered for a first period of time, and then subsequently withdrawn for a second period of time, wherein seizures are suppressed in the patient during the second period of time.

### BRIEF DESCRIPTION OF THE DRAWINGS

FIGS. 1A-E show EZH2 maintains seizure threshold after status epilepticus and mitigates chronic disease progression. (1A) Outline of experimental approaches for subsequent panels. (1B) In naïve mice, neuronal deletion of EZH2 does not alter flurothyl seizure threshold (unpaired t-test; n=13-16). (1C) After SE, seizure threshold was reduced by 26% in EZH2nHom mice, but was not altered in EZH2nWT mice (2-way ANOVA with the two-stage step-up method of Benjamini, Krieger, and Yekutieli (BKY); n=13-16). (1D) SRS activity was video recorded in epileptic EZH2nWT and nHom mice 5-7 weeks post-SE. EZH2nHom mice showed a 2.5-fold increase in seizure frequency (unpaired t-test; n=6-10). (1E) Total observed Racine scale events for EZH2nHom and EZH2nWT mice were quantified as a frequency distribution. EZH2nHom mice had more severe seizures than WT mice. (Kolmogorov-Smirnov test; D: 0.833; n=6-10).

FIGS. 2A-I show STAT3 drives hyper-activation of pro-inflammatory gene networks in neurons acutely post-SE in EZH2 knockout mice. (2A) Black Leiden cluster of 439 genes, one of twelve generated from 6241 differentially expressed genes (fold change >1.2×, adjusted p<0.01) across saline, kainate, EZH2nWT, and EZH2nHom conditions (see FIG. S3A). (2B) Ontological analysis of genes in the Black cluster highlighted inflammatory processes. (2C) MAGIC was used to determine which factors control the expression of Black cluster genes, and positive hits were projected as a Gene Regulatory Network (GRN). (2D) Top 5 factors driving expression in the Black cluster sorted by −log (adjusted p value). (2E, F) Factors were ranked by in degree and out degree counts, and betweenness centrality. (2G) Hippocampi were harvested on day 4 post-SE from EZH2nWT and nHom mice for western blot analysis. (2H) pSTAT3 was not altered by day 4 post-SE in EZH2nWT mice but was induced 4.5-fold in EZH2nHom mice (2-way ANOVA with Tukey's correction; n=7-12). (2I) N2A cells were stably transfected with EZH2 shRNA (shEZH2) or non-targeting shRNA (shNT). Knockdown of EZH2 caused induction of pSTAT3 and total STAT3 protein.

FIGS. 3A-F show pro-inflammatory genes activated in acute mouse epileptogenesis overlap with a module of genes in chronic human temporal lobe epilepsy. (3A) Comparison of 10 human TLE k-means gene clusters to the mouse Black cluster by Fisher Exact test. Plotting −log (adjusted p) versus odds ratio against the mouse Black cluster showed that the human Brown cluster is the most similar to the mouse Black cluster (Fisher exact; Odds: 4.79, p (adj)=1.2×10⁻²⁰). Dotted line shows p=0.05. (3B) Ontological analysis of genes in the human Brown cluster highlighted inflammatory processes. (3C) MAGIC GRN for Factors driving the human Brown cluster. (3D-E) Factors were ranked by their in degree, out degree, and betweenness centrality. (3F) STAT3 was the top driver predicted by MAGIC to drive expression of the 65 genes common to the mouse Black and human Brown clusters.

FIGS. 4A-F show JAK/STAT signaling is reactivated and targetable in chronic epilepsy. (4A) Representative western blots for pSTAT3, total STAT3, and tubulin in naïve and epileptic mice at 1 day, 4 days, and 14 weeks post-SE. (4B-C) pSTAT3 was induced 11-fold at 1 day and 7.3-fold at 14 weeks, but not induced 4 days post-SE. Total STAT3 was induced 1.4-fold on day 1 post-SE, but not 4 days and 14 weeks post-SE (2-way ANOVA with Tukey's correction; n=3-13). (4D) SRS activity was monitored from 5-9 weeks post-SE, and nHom mice received daily CP690550 injections from 7-9 weeks post-SE. (4E) CP690550 reduced seizure frequency by 80% in nHom mice, with nHom mice exhibiting seizure frequencies 58% below WT mice (1-way ANOVA with Tukey's correction; n=6-10). (4F) Total observed Racine scale events represented as a frequency distribution. Daily CP690550 reduced seizure severity in nHom mice compared to pre-treatment (Wilcoxon matched pairs signed rank; W: −36.00, p<0.01, n=6) and WT mice (Mann Whitney test; U: 119, p<0.05; n=6-10).

FIGS. 5A-G show CP690550 profoundly suppresses spontaneous seizures in epileptic wildtype mice. (5A) SE was induced via IHKA. Under continuous EEG recording, vehicle was administered daily for one week followed by one week of daily vehicle or CP690550 treatment. (5B) Vehicle-treated mice had seizures across both weeks of the experiment. (5C) Example power spectrum, trace, and seizure. (5D-E) Grouped comparisons for seizure frequency and burden were displayed as violin plots and cumulative frequency distributions (bin centers are marked). CP690550 treated mice showed a 13-fold reduction in seizure frequency and an 8-fold reduction in seizure burden (Kolmogorov-Smirnov test; D: 0.9091 (Frequency), D: 0.7083 (Burden); n=5-12). (5F-G) Heat maps display paired data for seizure frequency and burden. Except for one seizure observed on day 10, CP690550 eliminated seizures after two days of treatment.

FIGS. 6A-E show CP690550 enduringly suppresses spontaneous seizures in epileptic wildtype mice at least 2 months post-washout. (6A) SE was induced via systemic kainate, and behavioral seizures monitored by video from weeks 8-20 post-SE. Vehicle was administered from weeks 8-10 post-SE, followed by daily vehicle or CP690550 treatment weeks 11-12. After drug washout, video recording continued weeks 13-20 post-SE. (6B-C) Grouped comparisons for seizure frequency and burden were displayed as violin plots and cumulative frequency distributions (bin centers are marked). Mice treated with CP690550 showed a 14-fold reduction in seizure frequency and 22-fold reduction in burden (Kolmogorov-Smirnov test; D: 1.0 (for both frequency and burden); n=8-9). Paired seizure frequency (6D) and burden (6E) were displayed as violin plots ( [symbol] : CP-treated compared to pre-CP, #: vehicle compared to CP-treated) (2-way ANOVA with BKY correction; n=4-11). CP-treated mice showed an 8-fold reduction in frequency and 10-fold reduction in burden 2 weeks post-washout; 6 out of 9 mice (66%) were seizure-free during this time. Two months after drug washout, treated animals continued to exhibit a 29-fold reduction in seizure frequency and a 39-fold reduction in seizure burden compared to vehicle-treated mice.

FIGS. 7A-C show CP690550 rescues working and short-term memory deficits in chronically epileptic mice. (7A) Outline of experimental approaches for subsequent panels. (7B) Spatial working memory was severely impaired in untreated chronically epileptic mice; SA was reduced 35% from baseline at week 12 post-SE and 32% at week 14 post-SE. Mice treated with CP690550 showed a 31% rescue in SA at week 12 post-SE and a 37% rescue at week 14, compared to vehicle-treated mice (1-way ANOVA with BKY correction; n=7-37). (7C) Spatial short-term memory was likewise impaired in untreated mice; FA was reduced 52% from baseline at week 12 post-SE and 37% at week 14. In contrast, mice treated with CP690550 showed a 48% rescue in FA at week 12 post-SE compared to vehicle-treated mice (1-way ANOVA with BKY correction; n=7-23). Daily treatment with CP690550 rescued both SA and FA to levels indistinguishable from naïve mice both during treatment and after drug washout.

FIG. 8 shows the experimental strategy for valproic acid (VPA) as a model for drug-resistant epilepsy in mice.

FIG. 9 shows seizure frequency (as percent of baseline) after VPA treatment. The clinical threshold for VPA efficacy is a seizure reduction frequency ≥50%.

FIG. 10 shows CP690550 repressed seizure frequency in the VPA model of drug resistance.

FIG. 11 shows CP690550 repressed seizure burden in the VPA model of drug resistance.

FIG. 12 shows CP690550 restored memory in the spontaneous alternation test after failure with VPA.

FIG. 13 shows CP690550 restored memory in the forced alternation test after failure with VPA.

The above-described and other features will be appreciated and understood by those skilled in the art from the following detailed description, drawings, and appended claims.

### DETAILED DESCRIPTION

Of the studies that have profiled gene changes in the epileptic brain, nearly all observe robust activation of inflammation. Many pro-inflammatory pathways are rapidly induced with epileptic insult but are then quenched within days. In the brain, inflammation can be triggered by glial cells as well as neurons. Anti-inflammatory drugs have been used with varying degrees of success to suppress seizures in human patients, and some drugs targeting neuroinflammation in rodents have shown antiepileptogenic potential. However, the endogenous mechanisms that regulate neuroinflammation post-SE—acutely and chronically—are poorly understood. The inventors applied a systems approach to a transcriptomic consortium dataset that incorporated gene expression data across multiple laboratories and models of epilepsy. With this approach, the histone methylase Enhancer of Zeste Homolog 2 (EZH2) was identified as a potential driver of gene changes in dentate granule cells post-SE. EZH2 was induced by an order of magnitude across epilepsy models. Transient pharmacological inhibition of EZH2 post-SE worsened epileptic phenotypes, suggesting that EZH2 induction plays a protective role in epileptogenesis. In addition to EZH2, the analysis predicted Signal Transducer and Activator of Transcription 3 (STAT3) to be a driver of gene activation across models of SE, hinting at an interplay between EZH2 and STAT3 signaling.

Described herein are experiments demonstrating that an inflammatory gene network centered around STAT3 appears acutely after SE, and is negatively regulated by EZH2. STAT3 itself is activated robustly and quenched in an EZH2-dependent manner within days of SE. This is followed by an unexpected resurgent activation with spontaneous seizures in the chronic period. Inhibiting the first wave of JAK/STAT signaling has no antiepileptogenic effect. Targeting the second wave of STAT3 activation in chronic epilepsy with the JAK inhibitor CP690550 (tofacitinib), for example, results in an enduring, disease-modifying suppression of spontaneous seizures and the restoration of spatial memory. Similar results are expected for other JAK1 and JAK1/3 inhibitors. The treatment methods described herein are robustly disease modifying as the protective effects of the drug endure long after the drug has been withdrawn.

As used herein, epilepsy is defined as brain disorder that causes spontaneous, recurring seizures. A seizure is a sudden alteration of behavior due to a temporary change in electrical impulses in the brain. Seizures can cause a person to collapse, shake, become stiff, lose awareness and/or experience unusual sensations such as unusual smells or taste and tingling in the arms and legs. Typically, epilepsy is diagnosed when a patient has two or more unprovoked seizures (seizures of unknown cause) at least 24 hours apart. A patient suspected of epilepsy may be a patient having at least one seizure of unknown cause, sudden falls, muscle twitching, muscles becoming limp or rigid, unintentional urination, changes in awareness and behavior such as temporary confusion, loss of consciousness, staring, unusual behaviors, and the like.

Epilepsy includes genetic epilepsy and acquired epilepsy. Genetic epilepsies can be caused by a change in one or more genes as well as chromosomal changes. Genetic tests for epilepsy include chromosome arrays, epilepsy gene panel tests, exome sequencing, and the like. In contrast to genetic epilepsy, acquired epilepsy results from an insult to the brain resulting from brain injury or trauma, stroke, intracerebral hemorrhage, brain tumors, infectious disease, brain inflammation resulting from meningitis or encephalitis, status epilepticus, neurodegenerative disease, and the like. In many cases, epilepsy is idiopathic, that is, the insult causing epilepsy is unknown. Seizures can be observed shortly after the insult, or months or even years after the original insult. A patient suspected of epilepsy may be a patient having a genetic profile associated with epilepsy, or a patient who has had an insult or injury to the brain.

Epileptogenesis is defined as the process that links an initial insult to the brain to the propagation of spontaneous seizures. While the events from insult to epilepsy are not well-understood, it is believed that chemical, structural, cellular and molecular changes in the brain may contribute to the development of epilepsy.

Status epilepticus is defined as an unremitting bout of seizures lasting for more than 5 minutes, during which time the patient does not regain consciousness.

Many patients have drug-resistant epilepsy (also called drug refractory epilepsy), which is defined as epilepsy that does not respond to anti-seizure medications. Patients may be identified as having drug-resistant epilepsy when they fail to become seizure-free, e.g. have persistent seizures, after trying two different anti-seizure medications. About 10-15% of patients who fail to respond to their first anti-seizure medication respond to their second anti-seizure medication. Patient with drug-resistant epilepsy have higher rates of sudden unexpected death in epilepsy (SUDEP).

In an aspect, administering a Janus Kinase inhibitor, such as the Janus Kinase 1/3 inhibitor tofacitinib, suppresses seizures and/or rescues cognitive decline in patients with drug-resistant epilepsy. In specific aspects, administering the Janus Kinase inhibitor, e.g., Janus Kinase 1 or 1/3 inhibitor, to the patient reduces seizure burden, reduces seizure frequency, restores memory as evidenced by spontaneous alternations test, and/or restores memory as evidenced by forced alternation test. In an aspect, in the treatment of drug-resistant epilepsy, the Janus Kinase inhibitor, e.g., Janus Kinase 1 or 1/3 inhibitor, may be administered daily.

As used herein, anti-seizure medications include brivracetam (Briviact®), cannabidiol (Epidiolex®), carbamazepine (Carbatrol®, Tegretol®), cenobamate (Xcopri®), clobazam (Frisium®, Sympazan®), clonazepam (Rivotril®), divalproex (Depakote®), phenytoin (Dilantin®, Phenytek®), eslicarbazepine (Aptiom®), ethosuximide (Zarontin®), felbamate (Felbatol®), gabapentin (Neurontin®), ganaxolone (Ztalmy®), lacosamide (Vimpat®), lamogitrine (Lamictal®), levitracetam (Keppra®), methsuximide (Celontin®), midazolam, oxcarbazepine (Trileptal®), perampanel (Fycompa®), phenobarbitone (Phenobarb®), potiga (Ezogabine®), pregabalin (Lyrica®), primidone (Mysoline®), rufinamide (Banzel®, Inovelon®), tiagabine (Gabitril®), topiramate (Topamax®), vigabatrin (Sabril®), zonisamide (Zonegram®), and combinations thereof.

In an aspect, method of treating a patient with epilepsy or suspected of having epilepsy comprises administering a Janus Kinase inhibitor, e.g., Janus Kinase 1 or 1/3 inhibitor, to the patient. Exemplary patients include human patients and dogs, for example.

Unexpectedly, as described herein, the JAK 1/3 inhibitor tofacitinib resulted in sustained disease-modifying seizure suppression for weeks after the drug was withdrawn. This suppression of seizures was accompanied by the restoration of spatial memory. Similarly it is expected that more selective Janus Kinase 1 inhibitors such as upadacitinib (Rinvoq®) will have similar effects.

In an aspect, the Janus Kinase inhibitor, e.g., Janus Kinase 1 or 1/3 inhibitor, is administered for a first period of time, and is then subsequently withdrawn for a second period of time, wherein seizures are suppressed in the patient during the second period of time. In an aspect, during the first period of time, the Janus Kinase inhibitor, e.g., Janus Kinase 1 or 1/3 inhibitor, may be administered daily, weekly or monthly, preferably daily. During the second period of time, when the Janus Kinase inhibitor, e.g., Janus Kinase 1 or 1/3 inhibitor, is withdrawn, no Janus Kinase inhibitor, e.g., Janus Kinase 1 or 1/3 inhibitor, is administered. When the patient is a human patient, the first period of time can be 1 to 12 weeks, specifically 1 to 4 weeks, and more specifically 1 to 2 weeks. When the patient is a human patient, the second period of time can be 1 week to the lifetime of the patient, specifically 1 to 6 months, and more specifically 1 to 4 weeks.

If, after the second period of time, seizures re-emerge, the Janus Kinase inhibitor, e.g., Janus Kinase 1 or 1/3 inhibitor, will be re-administered and re-withdrawn.

In an aspect, the method further comprises, during the second time period, assessing behavioral and/or cognitive abilities in the patient. Without being held to theory, it is believed that Janus Kinase inhibitor, e.g., Janus Kinase 1 or 1/3 inhibitor, administration during the first period of time will improve the comorbid behavioral and cognitive decline in the patient typically observed with epileptic patients. Exemplary behavioral and/or cognitive abilities include spatial memory, cognitive dysfunction, depression, anxiety, schizophrenia, or a combination thereof.

In an aspect, when the patient is being treated for an autoimmune disease or atopic dermatitis, the patient has not previously been administered a Janus Kinase inhibitor, e.g., Janus Kinase 1 or 1/3 inhibitor. In another aspect, the patient is not being treated for an autoimmune disease or atopic dermatitis and/or has not been diagnosed with an autoimmune disease or atopic dermatitis.

Tofacitinib (Xeljanz®; 3-((3R,4R)-4-methyl-3-[methyl-(7H-pyrrolo[2,3-d]pyrimidin-4-yl)-amino]-piperidin-1-yl)-3-oxopropionitrile) is FDA approved for the treatment of rheumatoid arthritis, psoriatic arthritis and ulcerative colitis. Tofacinib citrate has the following structure:

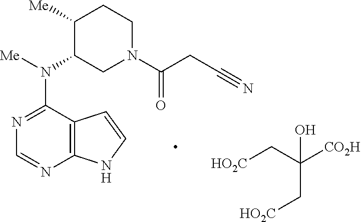 [SMILES: C[C@@H]1CCN(C(=O)CC#N)C[C@@H]1N(C)c1ncnc2[nH]ccc12 ; O=C(O)CC(O)(CC(=O)O)C(=O)O]

Xeljanz®, for example, is formulated as an immediate-release film-coated tablet for oral administration containing 5 mg of tofacitinib (equivalent to 8 mg tofacitinib citrate) or 10 mg of tofacitinib (equivalent to 16 mg tofacitinib citrate), in addition to excipients such as croscarmellose sodium, HPMC 2910/Hypromellose 6 cP, lactose monohydrate, macrogol/PEG3350, magnesium stearate, microcrystalline cellulose, titanium dioxide and triacetin. Xeljanz HR® is an extended-release film-coated tablet containing 11 mg of tofacitinib (equivalent to 17.77 mg tofacitinib citrate) in addition to excipients such as cellulose acetate, copovidone, hydroxyethylcellulose, hydroxypropylcellulose, and magnesium stearate.

Exemplary doses of tofacitinib are 5 mg to 10 mg administered two times per day (BID), or 11 mg in an extended-release form administered once per day. Doses of 10 times or more the standard daily doses can be tolerated. In an aspect, the tofacitinib is administered orally daily. In an aspect, the daily dosage of tofacitinib is 5 to 22 mg/day.

In an aspect, when the JAK inhibitor is tofacitinib, the patient is not being treated for an autoimmune disease such as rheumatoid arthritis, psoriatic arthritis, and ulcerative colitis.

Upadacitinib (Rinvoq®; (3S,4R)-3-Ethyl-4-(3H-imidazo[1,2-a]pyrrolo[2,3-e]pyrazin-8-yl)-N-(2,2,2-trifluoroethyl) pyrrolidine-1-carboxamide hydrate (2:1)) is currently indicated for the treatment of adults with moderately to severely active rheumatoid arthritis who have had an inadequate response or intolerance to one or more TNF blockers and other indications. Upadacitinib has been characterized in vitro as a JAK1 selective inhibitor (Parmentier et al., “In vitro and in vivo characterization of the JAK1 selectivity of upadacitinib (ABT-494)”, BMC Rheumatology (2018)2:23. Analysis of clinical study data (Mohamed et al., “Preferential inhibition of JAK1 relative to JAK3 by Upadacitinib: Exposure-response analyses of ex vivo data from 2 Phase I clinical trials and comparison to tofacitinib”, The Journal of Clinical Pharmacology (2020), 60 (2), 188-197) shows a potency ratio for inhibition of IL-6-induced pSTAT3 (measure of JAK1 activity) relative to IL-7-induced pSTAT5 (measure of JAK3 activity) of 2.0 compared to 0.67 for tofacitinib, indicating about a 3-fold higher selectivity for upadacitinib over tofacitinib for JAK1. While selective for JAK1, upadacitinib has significant JAK3 inhibitory activity.

Upadacitinib is available as oral extended-release tablets and an oral solution. A recommended daily dose of upadacitinib is 15 mg once daily in adults, and lower doses for children based on body weight. In some aspects, a loading dose of 45 mg once daily for 2-12 weeks can be followed by daily dosing at 15 mg once daily. Additional daily doses of upadacitinib include doses of 1 to 100 mg, specifically 6, 12, 18, 24 and 30 mg.

In an aspect, when the JAK inhibitor is upadacitinib, the patient is not being treated for atopic dermatitis or an autoimmune disease such as rheumatoid arthritis, ankylosing spondylitis, non-radiographic axial spondyloarthritis, psoriatic arthritis, ulcerative colitis, Crohn's disease, polyarticular juvenile idiopathic arthritis, giant cell arteritis, and the like.

Abrocitinib (Cibinqo®; N-((1s,3s)-3-(methyl (7H-pyrrolo[2,3-d]pyrimidin-4-yl)amino)cyclobutyl) propane-1-sulfonamide) is currently indicated for the treatment of patients with refractory, moderate-to-severe atopic dermatitis. In vitro, abrocitinib shows selectivity for JAK1 over JAK2 and JAK3. Abrocitinib is available as 50, 100 and 200 mg tablets. Additional daily doses of abrocitinib include doses of 1 to 1000 mg, specifically 25, 75, 125, 150, 250, 500 and 750 mg.

In an aspect, when the JAK inhibitor is abrocitinib, the patient is not being treated for atopic dermatitis or an autoimmune disease such as vitiligo, hand eczema, prurigo nodularis, alopecia areata, and the like.

Baricitinib (Olumiant®; {1-(ethylsulfonyl)-3-[4-(7H-pyrrolo[2,3-d]pyrimidin-4-yl)-1H-pyrazol-1-yl]azetidin-3-yl}acetonitrile) is currently indicated for the treatment of moderately to severely active rheumatoid arthritis and alopecia areata as well as COVID-19. In vitro, baricitinib shows selectivity for JAK1 and JAK2. Baricitinib is available as 1, 2 and 4 mg tablets for once daily dosing. Additional daily doses of baricitinib include doses of 0.5 to 10 mg.

In an aspect, when the JAK inhibitor is baricitinib, the patient is not being treated for an autoimmune disease such as rheumatoid arthritis, alopecia areata, and the like.

Filgotinib (Jyseleca®) is indicated for the treatment of moderate to severe active rheumatoid arthritis in adults who have responded inadequately to, or who are intolerant to one or more disease-modifying anti-rheumatic drugs. In vitro, filgotinib shows selectivity for JAK1 over JAK2 and JAK3. Filgotinib is generally dosed at 200 mg once daily. Additional daily doses of filgotinib include doses of 1 to 1000 mg, specifically 25, 50, 75, 100, 125, 150, 250, 500 and 750 mg.

In an aspect, when the JAK inhibitor is filgotinib, the patient is not being treated for an autoimmune disease such as rheumatoid arthritis, ulcerative colitis, and the like.

Ritlecitinib (Litfulo®) is indicated for the treatment of severe alopecia areata. In vitro, ritlecitinib shows specificity for JAK3. Ritlecitinib is available as a 50 mg oral capsule for once daily dosing. Additional daily doses of ritlecitinib include doses of 1 to 1000 mg, specifically 25, 75, 100, 125, 150, 250, 500 and 750 mg.

In an aspect, when the JAK inhibitor is ritlecitinib, the patient is not being treated for an autoimmune disease such as alopecia areata, and the like.

Additional inhibitors for use in the methods described herein are peficitinib (Smyraf®), decernotinib, and oclacitinib which inhibit both JAK1 and JAK3.

Peficitinib (Smyraf®) is approved in Japan for the treatment of rheumatoid arthritis. In an aspect, when the JAK inhibitor is peficitinib, the patient is not being treated for an autoimmune disease such as rheumatoid arthritis.

Decernotinib has not been approved, but has shown efficacy in relieving the symptoms of rheumatoid arthritis. In an aspect, when the JAK inhibitor is decernotinib, the patient is not being treated for an autoimmune disease such as rheumatoid arthritis.

Oclacitinib has not been approved for use in humans, but is used in dogs to treat atopic dermatitis. In an aspect, when the JAK inhibitor is oclacitinib, the patient is not being treated for atopic dermatitis.

In an aspect, the Janus Kinase inhibitor, e.g., Janus Kinase 1 or 1/3 inhibitor, is administered after an episode of status epilepticus. In another aspect, the patient has genetic epilepsy. In yet another aspect, the patient has acquired epilepsy. In a further aspect, the patient has drug-resistant epilepsy.

Exemplary drugs that can be co-administered with Janus Kinase inhibitor, e.g., Janus Kinase 1 or 1/3 inhibitor, include anti-seizure medications such as brivracetam (Briviact®), cannabidiol (Epidiolex®), carbamazepine (Carbatrol®, Tegretol®), cenobamate (Xcopri)®), clobazam (Sympazan®), divalproex (Depakote®), phenytoin (Dilantin®, Phenytek®), eslicarbazepine (Aptiom®), felbamate (Felbatol®), fenfluramine (Fintelpa®), gabapentin (Neurontin®), ganaxolone (Ztalmy®), lamogitrine (Lamictal®), Levitracetam (Keppra®), oxcarbazepine (Trileptal®), perampanel (Fycompa®), potiga (Ezogabine®), primidone (Mysoline®), rufinamide (Banzel®), tiagabine (Gabitril®), topiramate (Topamax®), vigabatrin (Sabril®), zonisamide (Zonegram®), and combinations thereof.

Drugs which have been used for the treatment of drug-resistant epilepsy include cannabidiol, cenobamate, felbamate, fenfluramine, perampanel, and combinations such as lamotrigine/valproic acid/topiramate, and the like.

Additional therapies for the treatment of drug-resistant epilepsy which can be combined with the methods described herein include a ketogenic diet, modified Atkins diet, low glycemic diet, vagal nerve stimulation, deep brain stimulation, surgery, and the like.

The invention is further illustrated by the following non-limiting examples.

### EXAMPLES

### Methods

Bioinformatics: Reads were mapped to the mm39 build mouse genome and subjected to Likelihood Ratio Tests using Deseq2. Transcripts per million were calculated and genes were classified as expressed if they had at least 3 TPM in all samples of any one of the 4 conditions (saline:WT, kainate:WT, saline:EZH2nHom, kainate:EZH2nHom). Deseq2 output was then filtered for only expressed genes and adjusted p-values were recalculated for the reduced number of genes. Genes with an adjusted p<.01 and 1.2-fold change were subjected to Leiden clustering with resolution=0.5 using the scanpy Leiden tool. Ontology was performed on all clusters using Ontomancer. Briefly, Biocarta, Hallmark, KEGG, and Reactome.gmt files were downloaded from Gene Set Enrichment Analysis (GSEA). Gene sets associated with every term in the databases were collected. For each Leiden cluster, Scores (Sₜ) for each term, t, were calculated as:

$${S}_{t} = \sqrt{{( log {O}_{t} )}^{2} + {( - log {P}_{t} )}^{2}}$$

Where Oₜ and Pₜ, are the odds ratio and p values from Fisher exact tests for the overlap of Leiden cluster genes and genes for each term t. Only terms with odds ratios>1 and p values<0.05 were kept for further analysis. For each term, a gene list is generated containing the overlap of genes in the cluster and term, Gₜ. A term x term similarity matrix was generated where similarity between term i and j is the Fisher exact test log odds ratio O_{i,j} for the intersection of genes in the terms Gᵢ and Gⱼ. This was converted to a network where nodes are terms. Edge weights were calculated as:

$$weight = {( log ( {O}_{i , j} ) / max log O )}^{6}$$

where max log odds is the maximum log odds value across all comparisons. The 6 power was used to separate nodes for visualization. Networks were generated using the scanpy Leiden tool with resolution=2. Each community was named after the highest scoring term in that community. Networks were visualized via Python's networkx and Plotly tools.

MAGIC was performed on all clusters essentially as described in the art but with r=1, and GTFs were ignored. GTFs were defined as any ENCODE Factors containing the strings “TBP”, “POL”, “GTF”, or “TAF”.

Gene Regulatory Network was projected using Cytoscape using MAGIC outputs. Factors were connected to their targets with edges defined as ChIP signal extracted from the Target Folder generated by MAGIC. Target genes of Factors that were not themselves Factors were removed to generate the GRN. Network statistics were calculated in Cytoscape.

Analysis of human TLE expression data: GSE63808 from GEO contained 129 transcriptomes from resected hippocampal TLE tissue. Probes with an Illumina® p-value<0.05 in at least 20% of samples were retained in the analysis; the probes were consolidated to gene symbols by taking the median probe expression value per symbol. These 9076 genes formed the background list for ontological and MAGIC analyses. Expression values were normalized by the median expression value in that sample. Genes kept for further analysis exhibited variance greater than the median coefficient of variance across all samples. The resulting 7658 unique genes were subjected to k-means clustering using the k-means tools from Sci-kit learn to divide genes into 10 clusters.

Overlap between each of the 10 human TLE clusters with mouse Black cluster genes was measured by Fisher exact test with the Benjamini-Hochberg correction for multiple comparisons. Ontological and MAGIC analyses were performed on the highest-scoring human Brown cluster as described in the previous section.

Cell culture: Growth conditions. Cells were grown in 5% CO₂ at 37° C. Neuro2a cells were grown in MEM (#10-009-CV Corning, Manassas, VA) with 1.0 g/L glucose, 1.5 g/L L-Glutamine, and 10% fetal bovine serum (#26140-079 Gibco, Grand Island, NY).

Lentiviral knockdown. Stable EZH2 knockdown in Neuro2a cells for western blot and RNA analysis was achieved using SMARTvector™ lentiviral delivery of shRNA per the manufacturer instructions. Puromycin selection began 48 hours after infection and maintained during cell expansion and experimentation. SMARTvector™ lentiviral mouse EZH2 mEF1a-TurboRFP shRNA (#V3SM7592-232015353, Dharmacon, Lafayette, CO) targeted the sequence ATCGTAGTAAGTACCAATG (SEQ ID NO: 1), and non-targeting mEF1a-TurboRFP control particles (#S10-005000-01) were used as an infection control.

Animal care: All animal procedures and experiments were performed with approval from the University of Wisconsin-Madison School of Medicine and Public Health Instructional Animal Care and Use Committee, or the Institutional Animal Care and Use Committee of Emory University, as appropriate, and according to NIH national guidelines and policies.

Transgenic mice. Cre-driver and EZH2 floxed mice were ordered from Jackson Laboratories (#003966, #022616, Bar Harbor, ME). Mice were bred on a C57BL6 background. Using the previously described Synapsin1-Cre system, EZH2^{-/-}; Syn1Cre^{+/-} (EZH2nHom) mice were generated by mating EZH2^{flox/flox}; Syn1Cre^{+/-} females with EZH2^{flox/flox} males. EZH2^{+/-}; Syn1Cre^{+/-} (EZH2nHet) mice were generated by mating EZH2^{flox/+}; Syn1Cre^{+/-} females with EZH2^{flox/flox} and EZH2^{flox/+} males. EZH2^{+/+}; Syn1Cre^{+/-} (EZH2nWT) mice were generated from mating pairs containing an EZH2^{flox/+} male. To assess Cre expression, a reporter mouse with a lox-P flanked STOP cassette preventing transcription of tdTomato (#007909 Jackson Laboratories) was used. Reporter males were bred with Syn1Cre^{+/-} females to generate Cre-positive animals and Cre-negative controls.

Repeated low-dose KA model. Male and female C57BL6 and FVB mice were bred and housed under a 12-hour light/dark cycle with access to food and water ad libitum. Mice were allowed to reach 4-6 weeks of age (C57BL6) 5-7 weeks of age (FVB) before undergoing experimentation. Kainate injections were performed at the same time of day (˜10 A.M.), and mice were returned to home cages before the start of the dark cycle (˜5 P.M.). Mice were housed with littermates for the duration of the experiment, with males and females separated at weaning.

A repeated low-dose kainate mouse model was used to induce SE. To begin, mice were weighed and singly housed in observation chambers for the duration of the injections. Mice were injected intraperitoneally (i.p.) with 5.0 mg/kg synthetic kainic acid (KA) dissolved in 0.9% saline (#7065, Tocris Bioscience, Bristol, United Kingdom). At twenty-minute intervals, mice were given 5.0 mg/kg injections of KA up to the third injection. The dosage was then reduced to 3.75 mg/kg. Animals continued to receive 3.75 mg/kg injections of KA every twenty minutes until each reached SE. The subsequent KA injection was skipped if an animal experienced two or more Class V seizures within a single twenty-minute interval. Injections resumed for the next round, unless the animal reached SE. During induction of SE, seizures were scored using a modified Racine Scale where I=freezing, behavioral arrest, staring spells; II=head nodding and facial twitches; III=forelimb clonus, whole-body jerks or twitches; IV=rearing; V=rearing and falling; VI=violent running or jumping behavior. Animals were considered to have reached SE after experiencing at least five Class V or VI seizures within a 90-minute window. KA mice were observed for 1-2 hours after SE. Animals were injected with 0.9% saline (s.c.), and soft gel food was provided in home cages. In the days following injection, animals were weighed and injected with 0.9% saline (s.c.) if body weight decreased by more than 0.5 g.

Pilocarpine model of status epilepticus. Adult C57BL6 mice (8-12 weeks old, Charles River) were injected with terbutaline (4 mg/kg ip, Sigma T2528) and methylscopolamine (4 mg/kg ip, Sigma S8502) to alleviate respiratory and cardiovascular effects of pilocarpine, then 30 min later received saline or pilocarpine (280 mg/kg i.p. as free base, Sigma P6503) as described in the art. After 1 hour of SE, seizures were interrupted by diazepam (10 mg/kg ip).

IHKA model of status epilepticus. The intrahippocampal kainic acid (IHKA) model was employed to generate chronically epileptic mice as previously described. Kainic acid (100 nL of 20 mM; vIHKA) or vehicle (100 nL of sterile saline; vIHSA) was stereotaxically injected into the hippocampus using the following coordinate (from Bregma): 3.6 mm posterior, −2.8 mm lateral, and 2.8 mm depth. Only mice which experienced status epilepticus and went on to develop spontaneous recurrent seizures were used in the current study.

Flurothyl and PTZ seizure threshold testing. Male and female C57BL6 mice were bred and housed under a 12-hour light/dark cycle with access to food and water ad libitum. Animals were allowed to reach 4-6 weeks of age before undergoing experimentation. Flurothyl seizure threshold tests were performed at the same time of day (˜10 A.M.), and mice were returned to home cages before 5 P.M. Mice were housed with littermates for the duration of the experiment, with males and females separated at weaning. All seizure threshold tests were video recorded, and seizure behavior was scored by a blinded observer. Two tests per mouse were performed: the first at one week prior to kainate injections and the second at four days post-SE.

Seizure threshold tests were performed on male and female EZH2nWT, EZH2nHet, and EZH2nHom mice using 100% bis(2,2,2-trifluoroethyl) ether (#287571, Sigma-Aldrich, Sₜ. Louis, MO). All tests were performed in a fume hood. Mice were placed in an airtight 10 L Plexiglas® chamber (8.5″×10.75″×6.75″). Flurothyl was infused into the chamber via a peristaltic pump at a rate of 40 μL/minute onto a piece of Whatman® filter paper placed at the top of the chamber. The time to generalized tonic clonic seizure (GTCS) was recorded from the start of flurothyl infusion. Mice reached GTCS when they exhibited a complete loss of postural and motor control. After experiencing a generalized tonic clonic seizure, mice were rapidly removed from the chamber, and flurothyl infusion was terminated. Mice were transferred to a recovery cage and kept isolated until motor control and normal behavior returned (˜10 minutes). Mice were returned to home cages after recovery. PTZ was dissolved at 5 mg/mL in 0.9% normal saline, and a single 45 mg/kg dose was administered (i.p.). Time to generalized tonic clonic seizure was recorded.

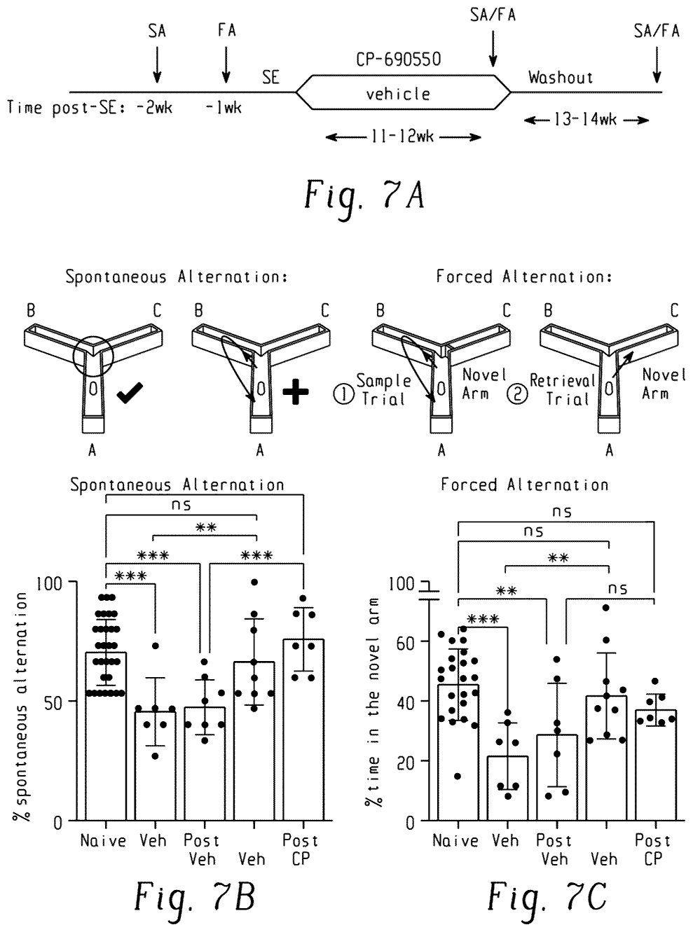

Long-term video monitoring for spontaneous seizures. Male and female FVB mice were bred and housed under a 12-hour light/dark cycle with access to food and water ad libitum. Animals were allowed to reach 5-7 weeks of age before undergoing kainate injections. Starting at either week 5 or week 7 post-SE, video recording for spontaneous seizures was performed at the same time of day (˜10 A.M.), and mice were returned to home cages before 5 P.M. Mice were singly housed in observation chambers for the duration of recording with access to food and water. During daily CP690550 treatment, mice were weighed and injected daily before recording began. Videos were reviewed and scored by an experimenter blinded to treatment and genotype using the modified Racine scale. In the experiment described by FIG. 7, only Racine class III-VI seizures were recorded. For each mouse, daily seizure burden was calculated as: (Total #of seizures*Average seizure duration(s))/(Total recording hours).

CP690550 citrate treatment of KA mice. Following kainate injections, an independent researcher (not the experimenter) randomly assigned mice to drug and vehicle conditions, and prepared both solutions. Drug and vehicle solutions were coded as “A” or “B.” CP690550 citrate (tofacitinib citrate) (#4556, Tocris Bioscience, Bristol, United Kingdom) was dissolved in a solution of 70% saline (0.9%)/15% ethanol/15% DMSO. The 70% saline/15% ethanol/15% DMSO solution was administered as vehicle. The independent researcher provided the blinded experimenter with the drug solutions and a list of all mice assigned to group A or B; this researcher did not participate in any seizure threshold testing or spontaneous seizure video scoring. In acute experiments, CP690550 or vehicle was delivered to mice intraperitoneally at 6, 18, and 24, 48, and 72 hours post-SE. The first dose of drug was given at 25 mg/kg, and all others were 15 mg/kg. In chronic experiments, CP690550 or vehicle was delivered intraperitoneally daily for two weeks at 15 mg/kg.

Continuous EEG recording and seizure detection. At the time of stereotaxic IHKA or IHSA injections, mice were implanted with chronic EEG recording headmounts (Pinnacle Technology, cat. #8201) which were affixed to the skull with 4 screws: 2 screws serving as EEG leads placed in the anterior and posterior cortex, 1 as a reference ground, and 1 as the animal ground. The headmounts were secured with dental cement. 24/7 EEG recordings were collected at 4 KHz using a 100× gain preamplifier high pass filtered at 1 KHz (Pinnacle Technology, cat. #8292-SE) and tethered turnkey system (Pinnacle Technology, cat. #8200). Seizures were detected using a custom analysis pipeline developed in-house in python as previously described in the art. Briefly, EEG traces were automatically detected using a thresholding method from traces that were downsampled (decimated) to 100 Hz and divided into 5 second segments. Power (2-40 Hz) and line length were used to identify seizures. Seizures were only included if two consecutive 5 second segments were classified as seizures (minimum 10 seconds length) and were manually verified by an experienced investigator with expertise in electrographic seizure detection. Seizure frequency was calculated as the #of seizures per day by measuring total number of seizures a mouse had over a 24-hour period. The average seizure duration was calculated by taking the mean seizure duration for each mouse in each recording period. For each mouse, seizure burden was calculated as: (Total #of seizures*Average seizure duration)/(Total EEG recording hours).

Y-maze cognitive testing. Working and short-term memory tests were performed at 3 time points: one (FA) or two (SA) weeks prior to induction of SE, post-CP690550 treatment (12 weeks post-SE), and post-washout (14 weeks post-SE). All tests were video recorded and analyzed by an experimenter blinded to treatment. Mice were habituated in the testing room for 30 minutes prior to all trials. Spontaneous alternation was used to evaluate working memory and forced alternation to evaluate short-term memory; both tests occurred within a three-arm Y-maze. For spontaneous alternation, mice were placed in the starting arm of the maze and allowed to explore freely for 8 minutes. Sequential arm choices during exploration of a three-arm Y-maze are considered an indicator of spatial working memory because the innate curiosity of a healthy mouse promotes exploration of the arm least recently visited. The number of choices resulting in a spontaneous alternation (e.g., an entry sequence of ABC but not ACA) was expressed as a percentage of the total number of choices.

After prolonged time in the Y-maze, habituation to novelty, and thus changes in motivation, can reduce the percentage of spontaneous alternations over time. Testing naïve mice, the transition from an exploratory phase to an escape phase was observed, and the total number of alternations for each mouse ranged from 20-100. To mitigate the effects of habituation and the variance in total alternation, only the first 15 alternations of each mouse were analyzed. Mice that did not reach 15 alternations were removed from the dataset. Distance traveled during each 8-minute test was measured to ensure that deficits in SA post-SE were not due to mobility impediments.

Forced alternation (FA) tests depend on a mouse's ability to recall and apply spatial short-term memory, motivated by their innate curiosity. FA tests involved two 5-minute trials 30 minutes apart: a sample trial and a retrieval trial. In the sample trial, the mouse was placed in the starting arm and allowed to explore the maze with one arm blocked off. Between trials the maze was washed with 70% EtOH. In the retrieval trial, the previously blocked arm was opened, and the mouse was allowed to explore freely for 5 minutes. Mice that did not enter all three arms of the maze during the retrieval trial of FA tests were excluded from the dataset. FA tests were quantified as percentage of time spent in the novel arm.

Tissue isolation and homogenization: After KA seizure induction and/or treatment with CP690550 citrate, animals were sacrificed by rapid decapitation. Whole hippocampal hemispheres were harvested and flash-frozen in liquid nitrogen or on dry ice. Hippocampal tissue was lysed in Radioimmunoprecipitation Assay Buffer (RIPA: 50 mM Tris, 150 mM NaCl, 1% Nonidet™ P-40, 0.5% sodium deoxycholate, 0.1% SDS) with mammalian protease inhibitor (1:1000, Sigma or 1860932 ThermoScientific) and phosphatase inhibitor (1:1000, Sigma or 78428 ThermoScientific). Protein from cell lines was harvested in Triton™ lysis buffer (3% 5M NaCl, 10% glycerol, 0.3% Triton™ X-100, 5% 1M Tris pH8.0) after PBS washes. Tissue was homogenized in lysis buffer by probe sonication (Fisher Scientific, Sonic Dismembrator, Model 100, Hampton, NH) on power 3 for two rounds, with 10 pulses per round. Supernatants were isolated by centrifugation and quantified using the DC Protein Assay (Bio-Rad, Hercules, CA) or BCA assay (ThermoScientific 23227). Protein extracts were stored at −80° C. 5× loading buffer (0.5 mM Tris, 10% SDS, 50% glycerol, 10 mM EDTA, and 1% beta-mercaptoethanol) was added to each sample to reach a 1× final concentration. Extracts in loading buffer were boiled at 95° C. for 5 minutes and stored for up to one month at −20° C. until run on an acrylamide gel.

Western blotting: Protein extracts in loading buffer were loaded at 20 μg per lane and resolved by electrophoresis in hand-poured acrylamide gels with a 5% acrylamide stacking layer (125 mM Tris pH6.8, 5% acrylamide, 0.01% ammonium persulfate, 0.01% SDS, 0.01% TEMED) and an 8% acrylamide separating layer (375 mM Tris pH 8.8, 8% acrylamide, 0.015% ammonium persulfate, 0.015% SDS, 0.08% TEMED). Gels were transferred to polyvinyl difluoride membranes (PVDF; Millipore, Bedford, MA) using Tris-glycine transfer buffer (20 mM Tris, 1.5 M glycine, 20% methanol). Membranes were blocked with 5% bovine serum albumin (Fisher Scientific, Fair Lawn, NJ) diluted in low-salt Tris-buffered saline (w/w TBST; 20 mM Tris pH7.5, 150 mM NaCl, 0.1% Tween® 20) with mammalian protease inhibitor (1:2000) and phosphatase inhibitor (1:2000), or Odyssey™ blocking solution (LI-COR, 927-60001) for 1 hour at room temperature. Primary antibodies were diluted in the same blocking buffer and incubated with membranes overnight at 4° C. Antibodies include: EZH2 (1:1000, #5426 Cell Signaling, Danvers, MA), phospho-STAT3 (Tyr705) (1:2000, #9145 Cell Signaling), total STAT3 (1:1000, #12640 Cell Signaling), JAK2 (1:1000, #3230 Cell Signaling), STAT1 (1:1000, #14994 Cell Signaling), pS727-STAT1 (1:1000, #8826 Cell Signaling), STAT2 (1:1000, #72604 Cell Signaling), pY1007/08-JAK2 (1:500, ab32101, Abcam, Cambridge, UK), Tubulin (1:1000, #T9026 Sigma-Aldrich), and Actin (1:10,000, #691001 MP Biomedicals). The next day, the membranes were washed three times in 1×TBST and incubated with horseradish peroxidase-conjugated goat-anti-mouse or -rabbit secondary antibodies for one hour at room temperature (1:10,000, Invitrogen #31430, #31460, Rockford, IL). Membranes were subsequently washed three times in TBST, and membranes were developed in SuperSignal™ West Femto ECL reagent (ThermoFisher, Waltham, MA). Bands were imaged using a ChemiDoc-It® Imaging System (UVP Vision-Works, Upland, CA) and quantified using UVP Vision Works software or ImageJ. Band intensities were graphed and analyzed using Prism 9 software (GraphPad Software, La Jolla, CA).

RNA extraction and clean-up for whole hippocampal RNAseq: Hippocampi were harvested from saline- and kainate-treated EZH2nWT and EZH2nHom mice on day 4 post-SE (n=4-5 per treatment/genotype). RNA was extracted from hippocampal tissue with Trizol® (#1559602, Life Technologies, Carlsbad, CA), and concentration was determined by spectrometry. This was followed by a treatment with DNase (#AM2238, Invitrogen). RNA clean-up was carried with the RNeasy® Mini Kit (#74104, Qiagen, Hilden, Germany). Purified samples were submitted to the Gene Expression core at the University of Wisconsin-Madison Biotechnology Center for library preparation and sequencing.

Immunofluorescence: Whole brains were harvested on day 1 or 4 post-SE, bisected sagitally, fixed in 4% paraformaldehyde, dehydrated in a series of graded TBS-Tween/ethanol solutions, embedded in paraffin, sectioned at 5 μm and subjected to immunofluorescence for NeuN, GFAP, total STAT3, and DAPI. Heat induced antigen retrieval was performed in a 10 mM citric acid buffer (pH 6.0). After a blocking step, mouse anti-NeuN (1:200 ab104224 Abcam, or 1:500 MAB377 from Millipore), rabbit anti-STAT3 (1:500 #12640 Cell Signaling), or rabbit anti-EZH2 (1:50 #5246 Cell Signaling) primary antibodies were applied and incubated overnight at 4° C. Slides were washed with PBS then incubated with goat-anti-mouse or goat-anti-rabbit Alexa Fluor® 488 (ab150113 Abcam or A11005, A11008 from In Vitrogen, respectively) and goat-anti-rabbit Alexa Fluor® 594 (ab150080 Abcam or Jackson ImmunoResearch) secondary antibodies at a concentration of 1:200 for one or two hours at room temperature, followed in some experiments by two-hour incubation at room temperature with Alexa Fluor® 488-conjugated mouse anti-GFAP antibody (1:200) (#53-9892-82 Invitrogen). Nuclear DNA was stained with DAPI (300 nM) or a DAPI-containing mounting media (#H-1200 Vector Laboratories, Burlingame, CA). Slides were imaged at 20-40× magnification using a fluorescent microscope (Nikon E600 microscope with fluorescence or Zeiss Axio Observer A1 epifluorescence microscope). Images were captured with Zeiss Axiovision or Cellsens software (Olympus Life Science Solutions, Center Valley, PA). Image analysis and processing were performed in ImageJ.

Statistical analysis: For all statistical analysis—with the exception of RNAseq data—q<0.05 or p<0.05 corrected for multiple comparisons was considered statistically significant (*p/q<0.05, **p/q<0.01, ***p/q<10⁻³, ****p/q<10⁻⁴). Data were graphed as mean±standard deviation (SD) in Prism 9 software (Graphpad Software, La Jolla, CA). Statistical tests were also performed in Prism. Western blot, seizure threshold, and spontaneous seizure data was analyzed by t-test, 2-way ANOVA, or 1-way ANOVA, with Tukey's correction for multiple comparisons or the two-stage step-up method of Benjamini, Krieger, and Yekutieli. Cumulative frequency distributions were measured by Kolmogorov-Smirnov tests for unpaired data, and the Wilcoxon matched pairs signed rank test for paired data. All statistical tests were two-tailed.

### Example 1: Neuronal EZH2 Maintains Seizure Threshold and Protects Against Exacerbated Epileptic Phenotypes

The histone methyltransferase EZH2 was previously identified as a potential driver of gene repression post-SE, and EZH2 protein was robustly induced over a 10-day window post-SE in rats and mice across models of epilepsy. EZH2 induction was protective because pharmacological inhibition resulted in accelerated disease progression. To determine the role of EZH2 in epilepsy, EZH2 floxed mice were crossed with Syapsin1 Cre drivers to delete EZH2 in mature neurons. Neuronal specificity of Cre expression was confirmed via crossing Syn1Cre with flox-STOP-flox-tdTomato mice and immunofluorescence (data not shown).

Immunofluorescence and western blot analysis confirmed successful ablation of neuronal EZH2 post-SE. Diminished induction of EZH2 was demonstrated on day 4 post-SE in mice lacking one EZH2 allele in neurons (EZH2nHet) or lacking both alleles (EZH2nHom) (data not shown). Staining for EZH2 confirmed reduced induction of neuronal EZH2 in EZH2nHet versus wildtype (EZH2nWT) mice (data not shown).

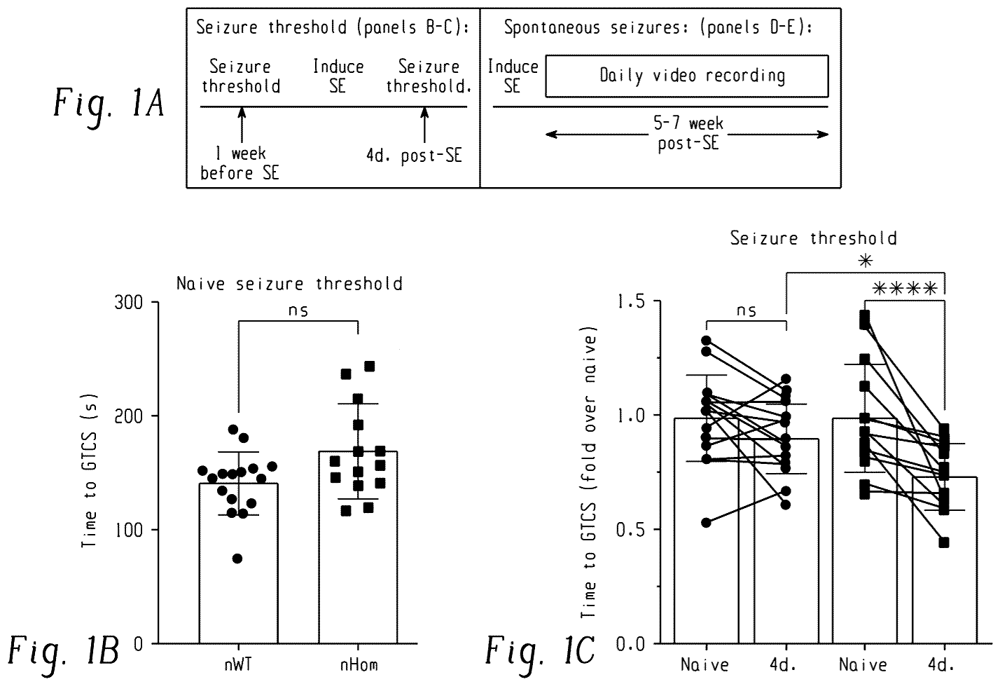

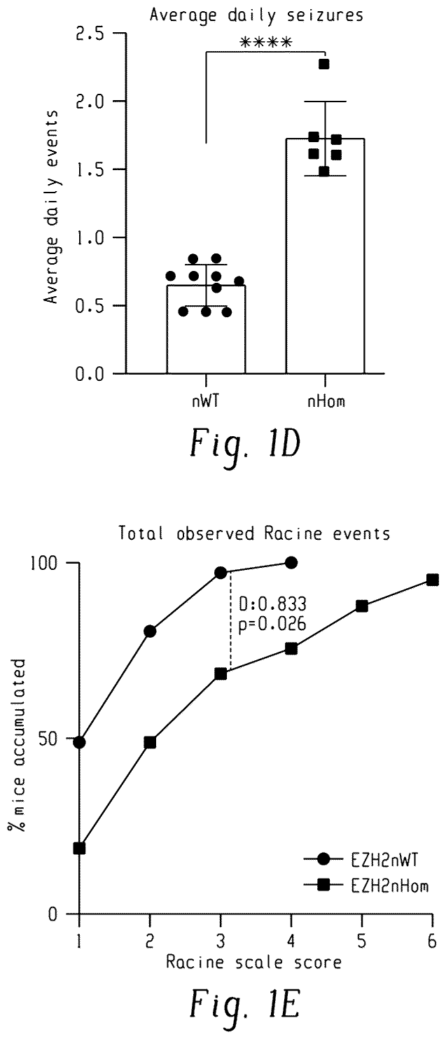

The impact of EZH2 deletion on flurothyl seizure threshold acutely post-SE was tested as a correlation between flurothyl seizure threshold and the development of spontaneous seizures has been suggested. Time to generalized tonic clonic seizure (GTCS) after exposure to the volatile chemoconvulsant flurothyl was measured in EZH2nWT and EZH2nHom mice aged 4-6 weeks, before and after kainate-induced SE (FIG. 1A). Deletion of EZH2 in neurons had no impact on seizure threshold in naïve mice (FIG. 1B), and induction of SE did not alter the seizure threshold of EZH2nWT mice on day 4 post-SE (FIG. 1C). In contrast, robust reductions in threshold were detected after SE in EZH2nHom mice (2-way ANOVA; 26% reduction, p<10⁻⁴). These were significant reductions compared to EZH2nWT mice. These data suggest that EZH2 is required to maintain seizure threshold post-SE.

The possibility that EZH2 loss primed the mice to experience more severe responses to kainate injection, and it was this vulnerability to SE driving the observed reduction in mutant seizure threshold. However, no differences in time to first class V seizure, and no correlation between time to first class V seizure and time to GTCS post-SE for either EZH2nWT (Pearson correlation; R²: 0.016, p=0.633) or EZH2nHom mice (R²: 0.152, p=0.209) was observed (data not shown).

To test the impact of neuronal EZH2 deletion on chronic disease, behavioral spontaneous seizures were monitored in FVB EZH2nWT and EZH2nHom mice by video from weeks 5-7 post-SE (FIG. 1A). EZH2nHom mice exhibited 2.5-fold greater seizure frequency compared to EZH2nWT mice (unpaired t-test; p<10⁻⁴) (FIG. 1D). EZH2nHom mice also exhibited more severe Racine scale seizures than EZH2nWT mice (Kolmogorov-Smirnov test; D: 0.833, p<0.05) (FIG. 1E). These data support the hypothesis that neuronal EZH2 induction post-SE is a protective response.

### Example 2: EZH2 Tempers Activation of the JAK/STAT Pathway Acutely Post-SE

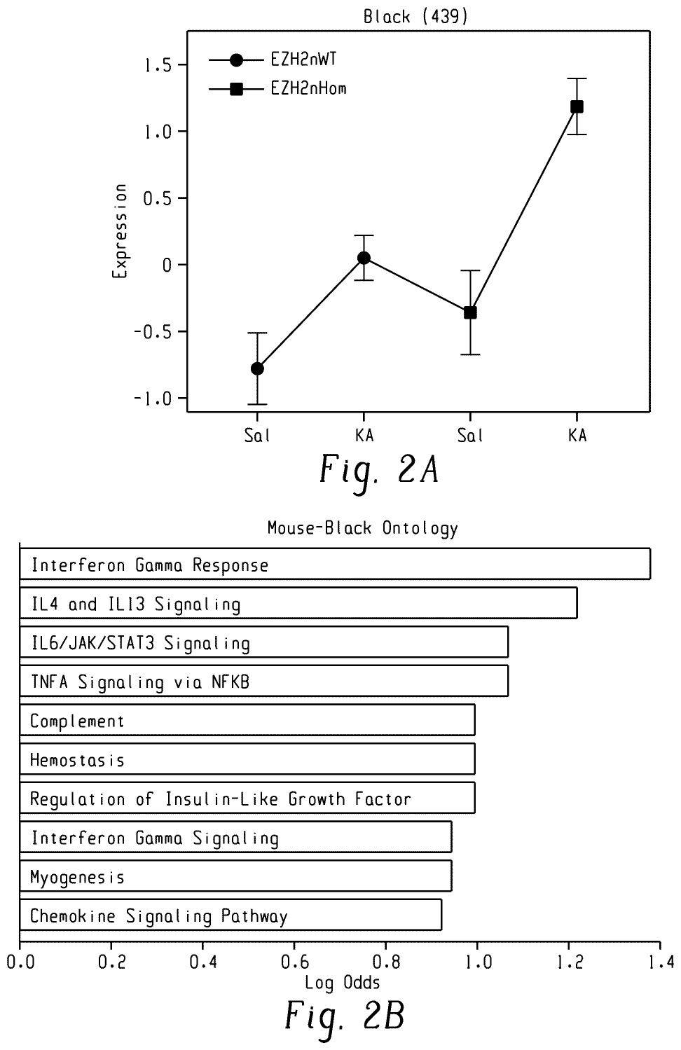

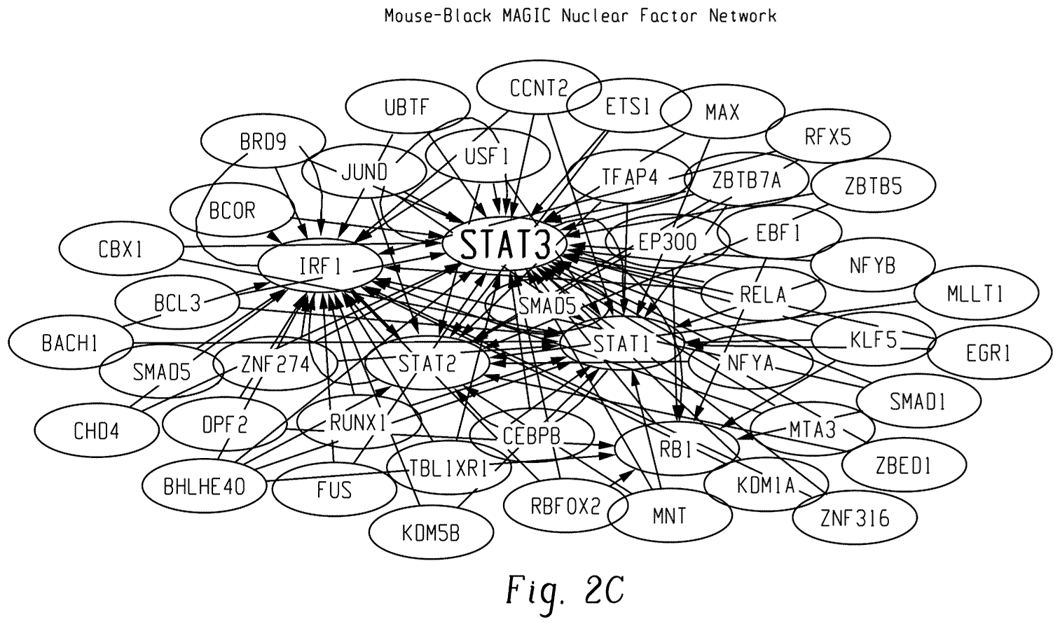

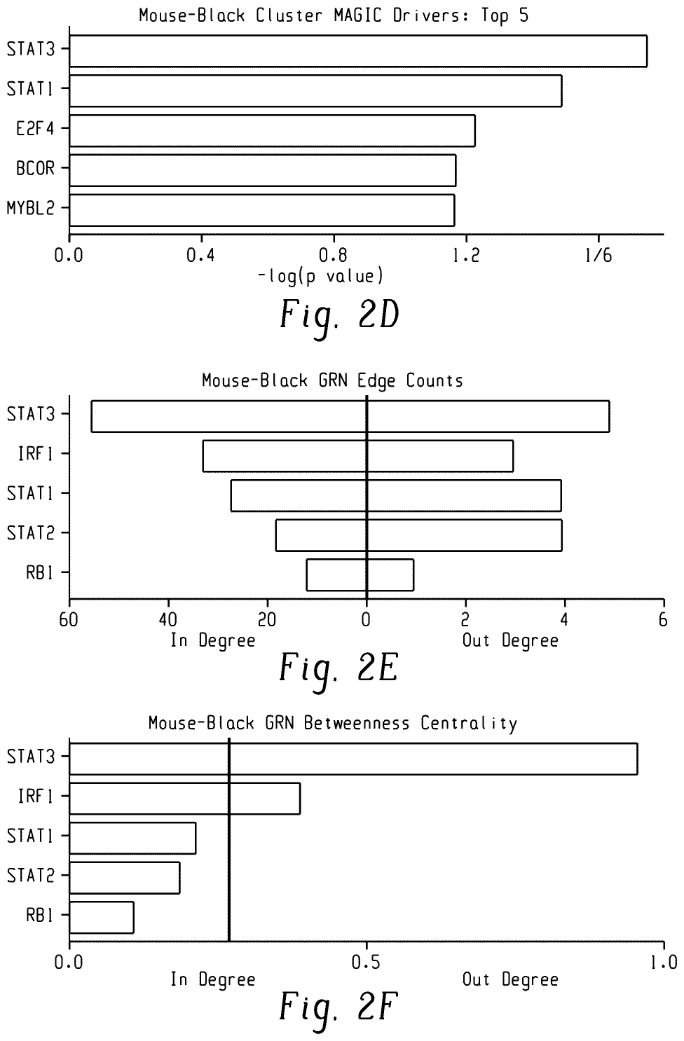

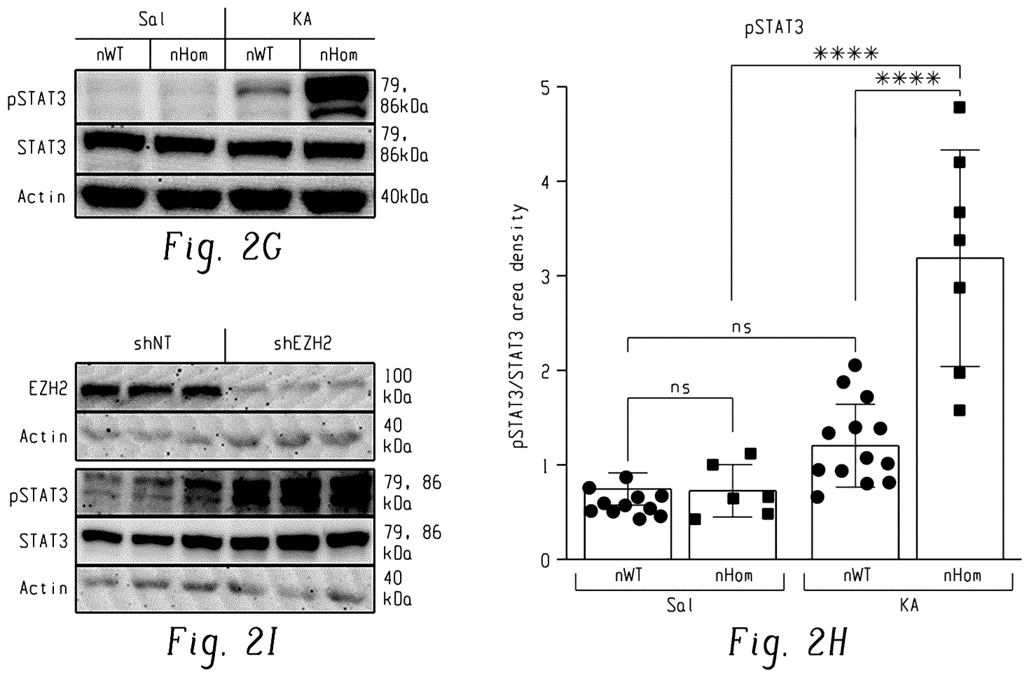

To begin to understand the mechanism behind EZH2-mediated protection, bulk RNAseq was performed on whole hippocampal tissue from naïve and epileptic (4d. post-SE) EZH2nWT and EZH2nHom mice. Of the 10556 expressed genes, 6241 were found to be differentially expressed at an adjusted p<10⁻² (Likelihood Ratio Test) across the four conditions (saline, kainate, EZH2nWT, EZH2nHom). A community-based network approach was used to group genes into clusters. Leiden clustering generated 12 distinct groups. Six clusters were generally increased, and 6 were generally decreased with SE. One cluster, (the Black cluster) which was generally increased, was selected because these 439 genes were induced with SE in EZH2nWT mice but manifested an exaggerated induction with SE in EZH2nHom mice (FIG. 2A). Without being held to theory, it was hypothesized that these were genes whose expression is induced but tempered in the presence of EZH2 post-SE, and which undergo unfettered induction in the absence of EZH2.

To understand the nature of genes comprising the Black cluster, an ontological analysis was performed. The 439 Black cluster genes were compared to gene lists associated with terms in the KEGG, Reactome, Biocarta and Hallmark databases. (FIG. 2B). The ontology network (data not shown) highlights the innate immune response (NFkB, Interferon Gamma, Complement) and inflammation (IL6 Jak Stat3) amongst others. The term ‘IL6 Jak Stat3’ was of particular interest given that previous work highlighted STAT3 as a driver of gene activation across models of epilepsy, and others have identified a pathological role for the JAK/STAT pathway in epileptogenesis.

To determine which transcription factors and cofactors (hereafter together referred to as ‘Factors’) control expression of Black cluster genes, MAGIC was used. STAT3 was the highest scoring Factor (FIG. 2D). When projected as a Gene Regulatory Network (GRN) (FIG. 2C) such that an arrow projects from Factor X to Factor Y if the gene encoding Factor Y is a transcriptional target of Factor X, STAT3 stood out as a central node. Thus, STAT3 had the highest in-degree, out-degree, and total edge count (FIG. 2E) of any Factor in the network.

Finally, the Betweenness Centrality of the network was assessed to determine which Factors might be critical for information flow in the GRN. STAT3 had by far the largest Betweenness Centrality of any Factor (FIG. 2F).

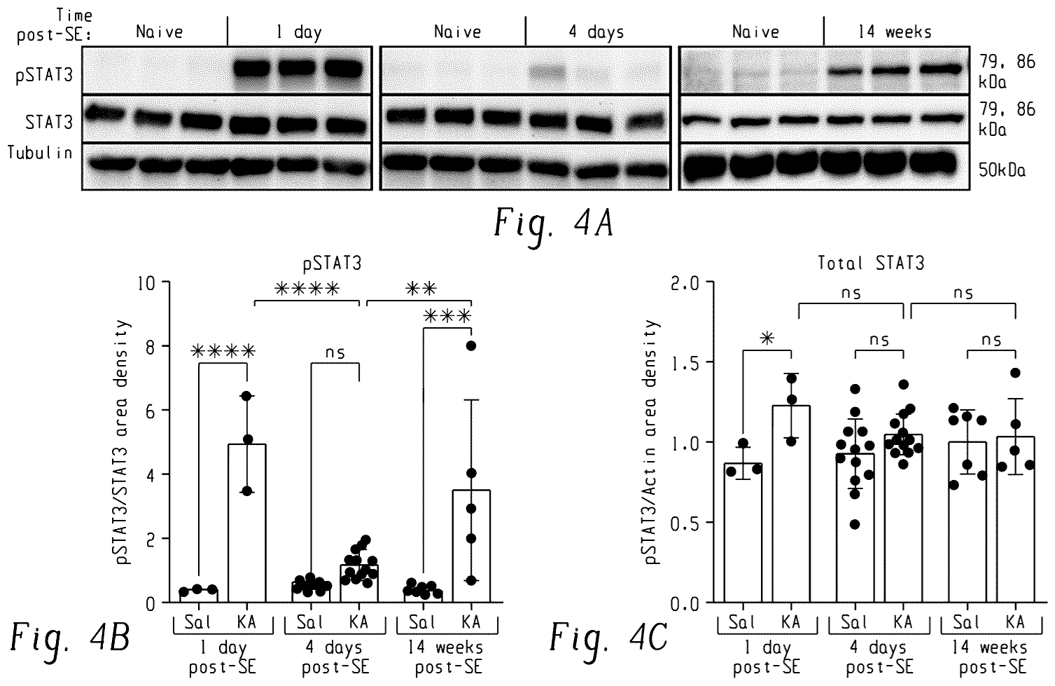

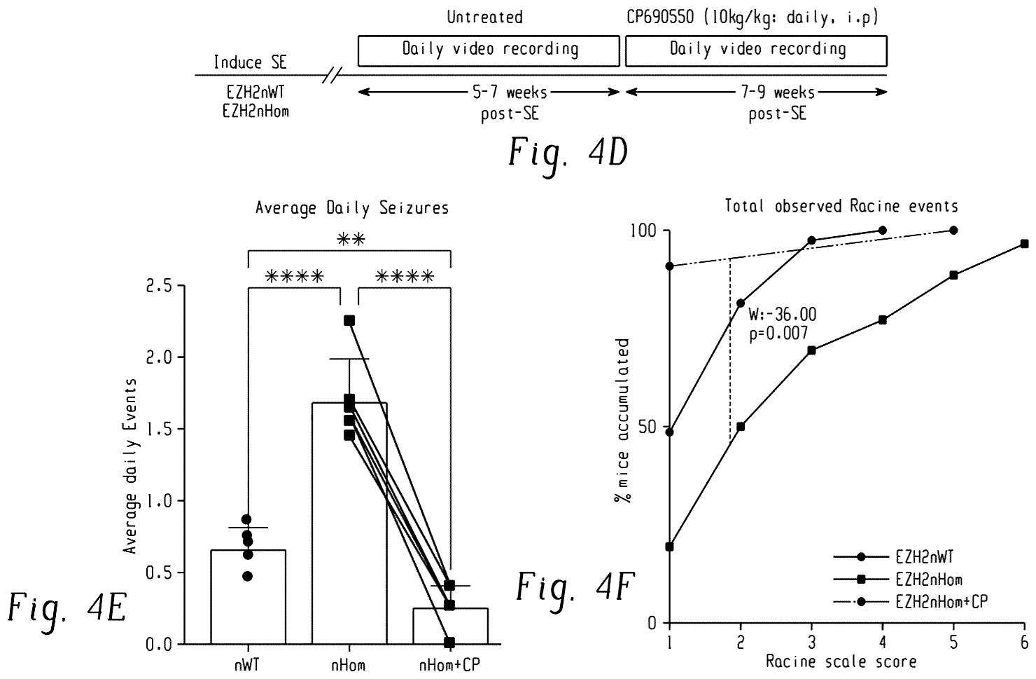

Activation of the JAK/STAT pathway acutely post-SE in WT mice has been reported by others (and FIG. 4A-C, data not shown). To test for a neuronal EZH2-dependent change in JAK/STAT signaling, a western blot analysis was performed on hippocampal lysates from 4d post-SE WT and EZH2 mutant mice (FIG. 2G). Having undergone a burst of activation on day 1 post-SE (see FIG. 4A), by day 4 post-SE, phosphorylation of STAT3 at Tyr705 (pSTAT3) was almost back to baseline in EZH2nWT mice compared to naïve controls. In contrast, EZH2nHom mice exhibited robust, sustained pSTAT3 induction in the absence of changes to total STAT3 (2-way ANOVA; 4.5-fold, p<10⁻⁴) (FIG. 2G-H, data not shown). These results demonstrated an EZH2-dependent suppression of STAT3 activation in the days following SE.

To define the cell types in which STAT3 is activated after seizures, immunofluorescence was performed with DAPI, NeuN, and total STAT3 antibodies on day 1 post-SE hippocampi taken from mice injected with saline or kainic acid (data not shown). The fact that total STAT3 protein is induced with STAT3 activation on day 1 post-SE was used (FIG. 4C), and so elevated total STAT3 protein was a proxy for activated STAT3. In both the dentate granule and CA1 pyramidal cell layer, a robust induction of total STAT3 in NeuN-positive cells was observed.

STAT3 was a central factor in the Black cluster GRN whereas, surprisingly, EZH2 was not. To understand how STAT3 hyperactivation occurred with EZH2 deletion, shRNA was used to knock down EZH2 in the Neuro2A (N2A) neuronal cell line. Robust diminution of EZH2 was observed in knockdown cells (shEZH2) (FIG. 2I, data not shown) (unpaired t-test; 3.7-fold, p<10⁻⁴). This was associated with a marked increase in pSTAT3 levels (unpaired t-test; 2.4-fold p<10-+) (data not shown). This result suggested that EZH2 suppresses STAT3 activation in a cell-autonomous manner without circuit level alterations.

### Example 3: JAK/STAT Signaling is Reignited in Chronic Epilepsy

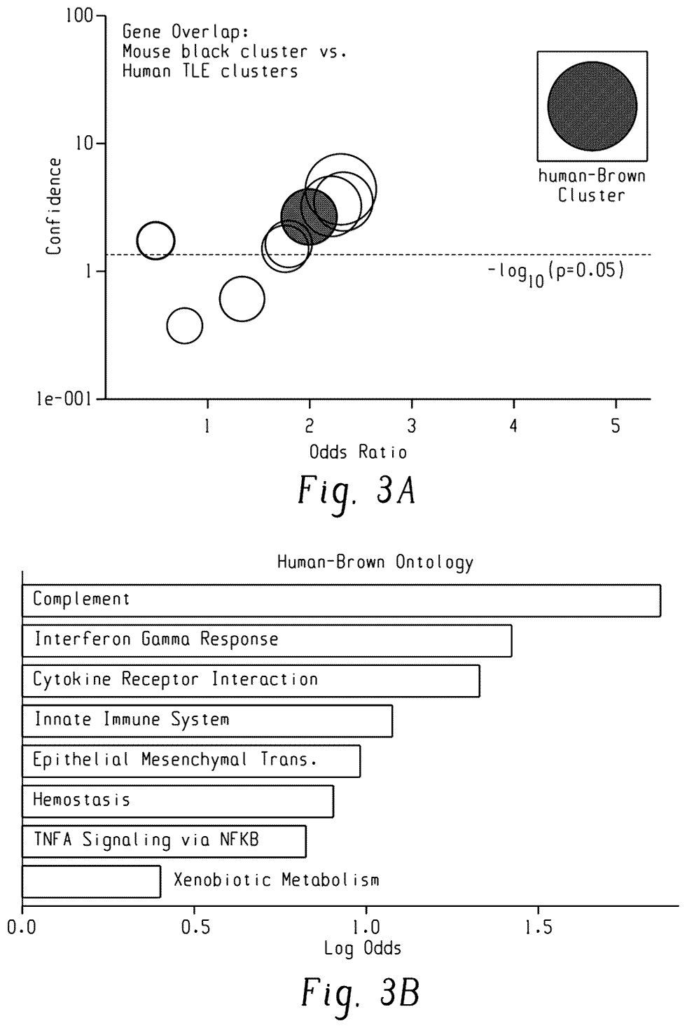

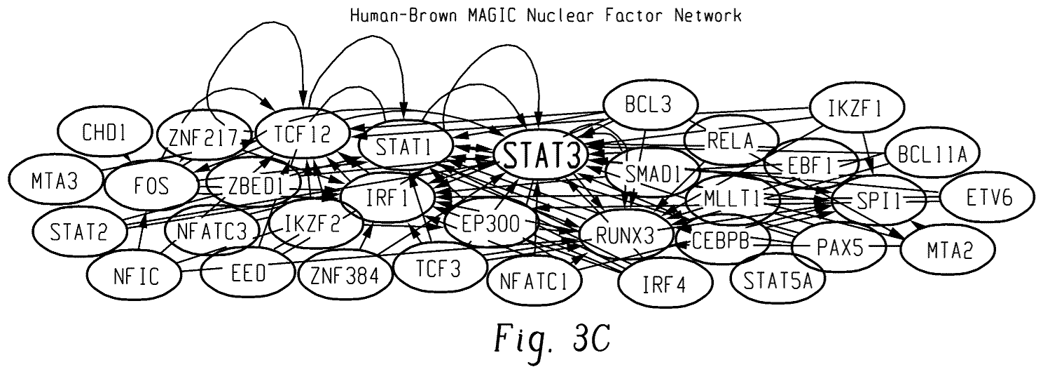

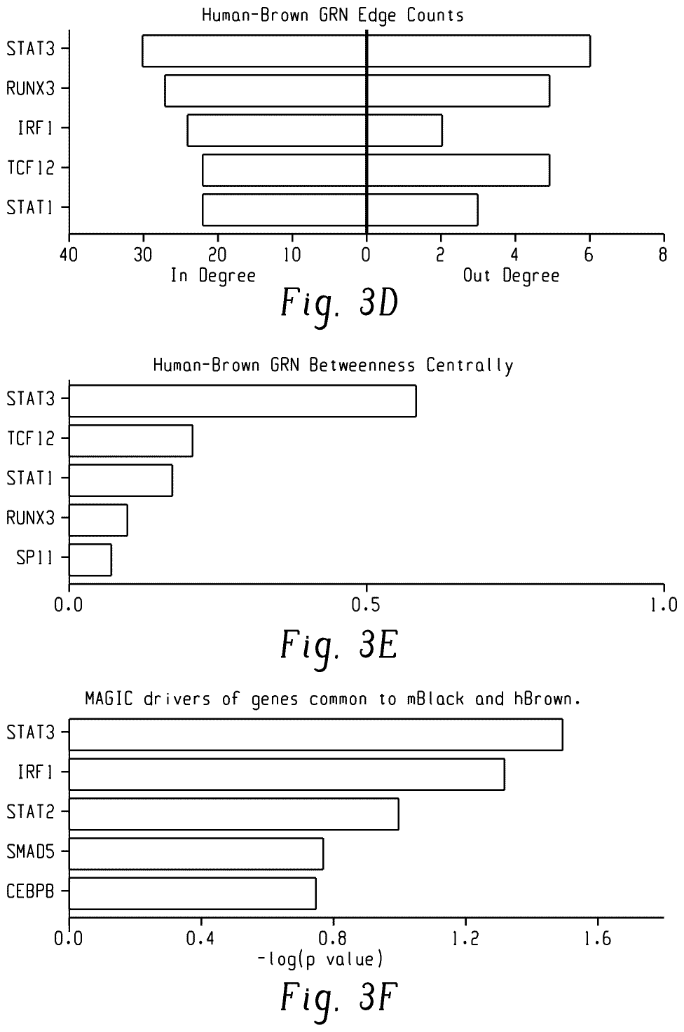

To ascertain whether the mouse Black cluster has a counterpart in human epilepsy, the whole transcriptome from 129 ante-mortem human temporal lobectomy patients was used. K-means clustering was used to generate 10 gene clusters from the 7658 genes co-expressed across all samples. To test whether any of these human clusters corresponded to the mouse Black cluster, Fisher exact tests were performed to measure the degree of overlap between the 439 mouse Black cluster genes, and the genes comprising each cluster of the human TLE dataset (FIG. 3A). The human Brown cluster showed the greatest overlap with the mouse Black cluster (Fisher exact; Odds: 4.79, p=1.19×10⁻²⁰, 65 gene overlap). Ontological analysis highlighted terms broadly similar to the mouse Black cluster such as inflammation (Cytokine Receptor Interaction/Complement/Innate Immune System) and interferon signaling (Interferon Gamma Response). Mirroring results of the mouse Black cluster, STAT3 in the human Brown GRN had the highest in-degree, out-degree, and betweenness centrality of any Factor in the network (FIG. 3C-E). MAGIC called STAT3 as the highest scoring Factor driving expression of the co-expressed genes (FIG. 3F). Given that the mouse Black cluster is from samples harvested 4 days post-SE and the Brown cluster is from chronically epileptic humans, this data suggests that STAT3-mediated disease mechanisms that are activated acutely in epileptogenesis are present in chronic epilepsy.

Following status epilepticus, robust activation of the JAK/STAT pathway diminishes within days (FIG. 2G), yet MAGIC called STAT3 as the top factor driving gene expression in the chronically epileptic human Brown cluster. The hypothesis that STAT3 activation reignites in chronic epilepsy was tested. Hippocampi were collected from mice at 1 day, 4 days, and 14 weeks post-SE (FIG. 4A). On day 1 post-SE, pSTAT3 was induced 11-fold over naïve levels (2-way ANOVA; p<10⁻⁴), but levels were diminished by day 4 post-SE (FIG. 4B, data not shown). After the onset of spontaneous seizures at 14 weeks post-SE, we observed robust activation of STAT3 (7.3-fold, p<10⁻³). This reactivation of JAK/STAT signaling corroborated the high degree of overlap between the pro-inflammatory acute mouse Black and chronic human Brown clusters. JAK/STAT3 inflammatory signaling is transiently activated in neurons and glia after SE, abates over a period of days, but undergoes reignition with spontaneous seizures.

### Example 4: JAK/STAT Signaling is a Potent Target in Chronic Epilepsy

To screen for potential drugs that could suppress STAT3 activation and reignition, the constitutive STAT3 activation in shEZH2 cells was used (FIG. 2I). shNT and shEZH2 N2A cells were incubated with either WP1066 (an analogue of the JAK2 inhibitor AG490) or CP690550 citrate (a small molecule inhibitor of JAK1/3 kinase activity). WP1066 did not impact pSTAT3 in shEZH2 cells at any concentration tested (data not shown). In contrast, 500 nM CP690550 citrate reduced pSTAT3 levels in shEZH2 cells to that of vehicle-treated shNT cells (2-way ANOVA; 2.3-fold, p<0.01) (data not shown).

It was determined whether CP690550 could rescue the increased seizure burden of EZH2nHom mice if administered during the chronic period. Spontaneous seizures were recorded from weeks 5-7 post-SE in EZH2nWT and nHom mice, followed by another 2-week recording period during which the EZH2nHom mice received daily injections of CP690550 (FIG. 4D). Target engagement was confirmed by western blot (data not shown).

Daily CP690550 treatment in the chronic period alleviated seizure severity in EZH2nHom mice (Wilcoxon paired; W: −36.0, p<0.01) and reduced seizure frequency by 80% (1-way ANOVA; p<10⁻⁴) (FIG. 4E-F). Indeed, seizure frequency of drug-treated EZH2nHom mice was reduced to below EZH2nWT levels (1-way ANOVA; 1.9-fold, p<0.01). Given this robust seizure suppression when administered during the chronic period, the possibility that CP690550 may simply be an anticonvulsant was considered. To test this, seizure threshold was measured using both flurothyl and PTZ (pentylenetetrazole) in mice pre-treated with vehicle or CP690550 (15 mg/kg, i.p.) thirty minutes prior to testing. This time was chosen because peak plasma levels are observed 30 to 60 minutes after oral administration. CP690550 pre-treatment had no effect on flurothyl or PTZ seizure threshold (data not shown).

### Example 5: CP690550 Profoundly Suppresses Electrographic and Behavioral Spontaneous Seizures in Chronically Epileptic Wildtype Mice Across Models

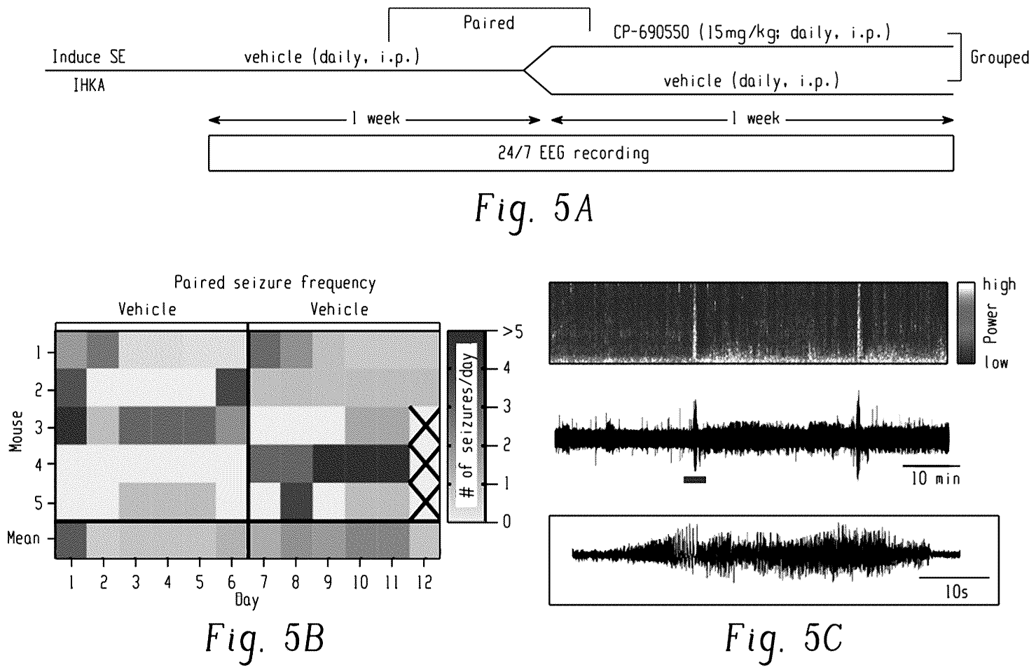

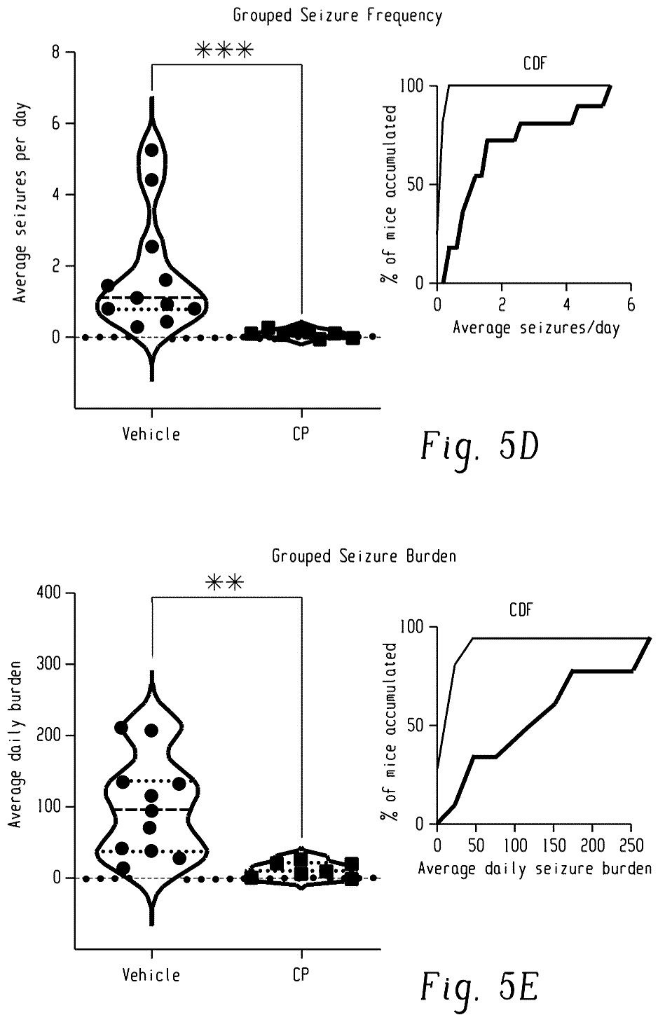

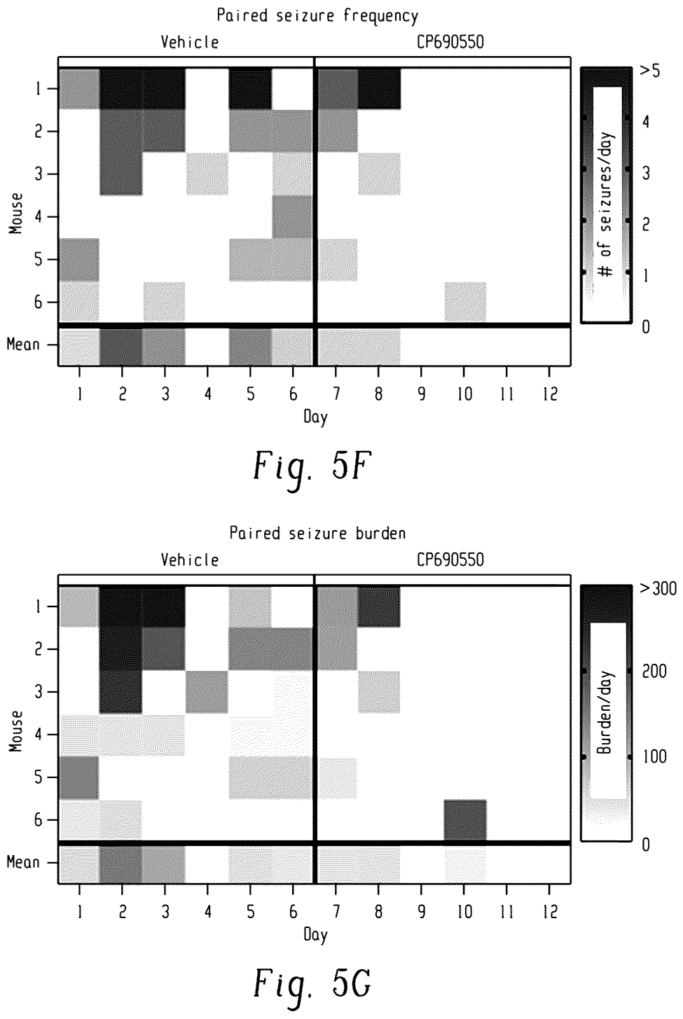

CP690550 treatment during the chronic period reduced seizure frequency in EZH2nHom mice to below wildtype levels, its effect on WT epileptic mice was tested. Wildtype mice were made epileptic via intrahippocampal kainate injections. In a paradigm involving one week of daily vehicle (i.p.) administration followed by an additional week of either vehicle or CP690550 injections (15 mg/kg, i.p.) under continuous EEG recording (FIG. 5A), CP690550 treatment during the chronic period profoundly suppressed spontaneous seizures. CP690550-treated mice showed a 13-fold reduction in seizure frequency (Kolmogorov-Smirnov test; D: 0.91, p<10⁻³) and 8-fold reduction in burden (Kolmogorov-Smirnov test; D: 0.71, p<0.01) over 1 week of recording (FIG. 5D-E). CP690550 treatment robustly reduced both spontaneous seizure frequency and burden in epileptic wildtype mice.

Additionally, while all animals experienced seizures during vehicle treatment, 80% of mice were seizure-free by the third day of CP690550 treatment (FIG. 5F-G). Animals treated with vehicle exhibited seizures throughout the experiment (FIG. 5B), contrasting with the profound reduction observed during CP690550 treatment.

Next the therapeutic effect of CP690550 in the systemic kainate model was tested using video-based behavioral seizure monitoring. These experimental modifications aimed to generalize the efficacy of CP690550 across epilepsy models and laboratories, minimize potential confounding effects introduced by headmount implantation, and prioritize clinically relevant seizure endpoints.

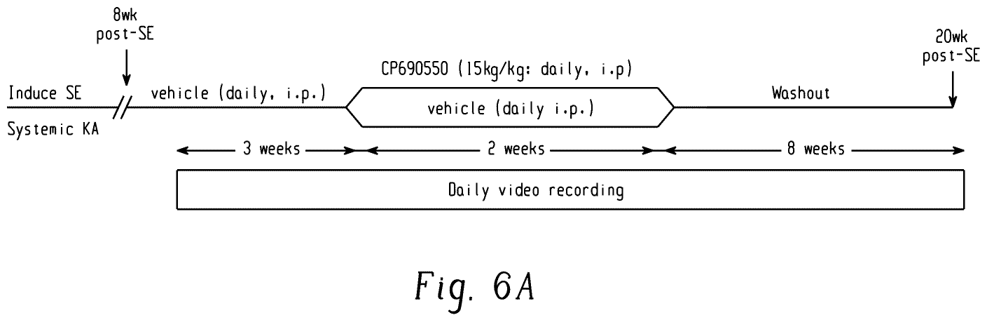

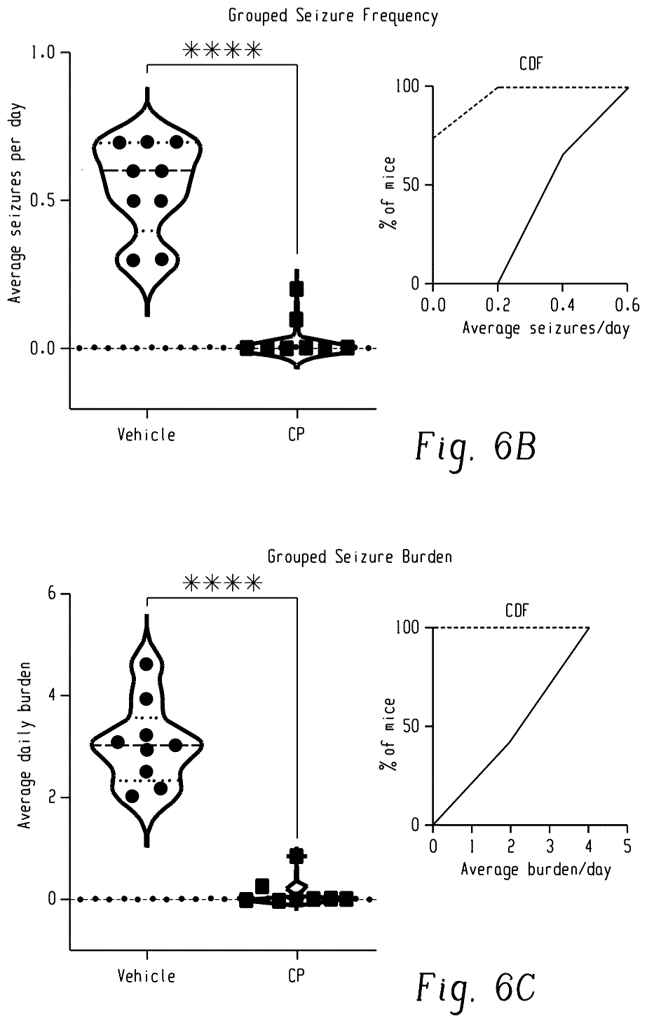

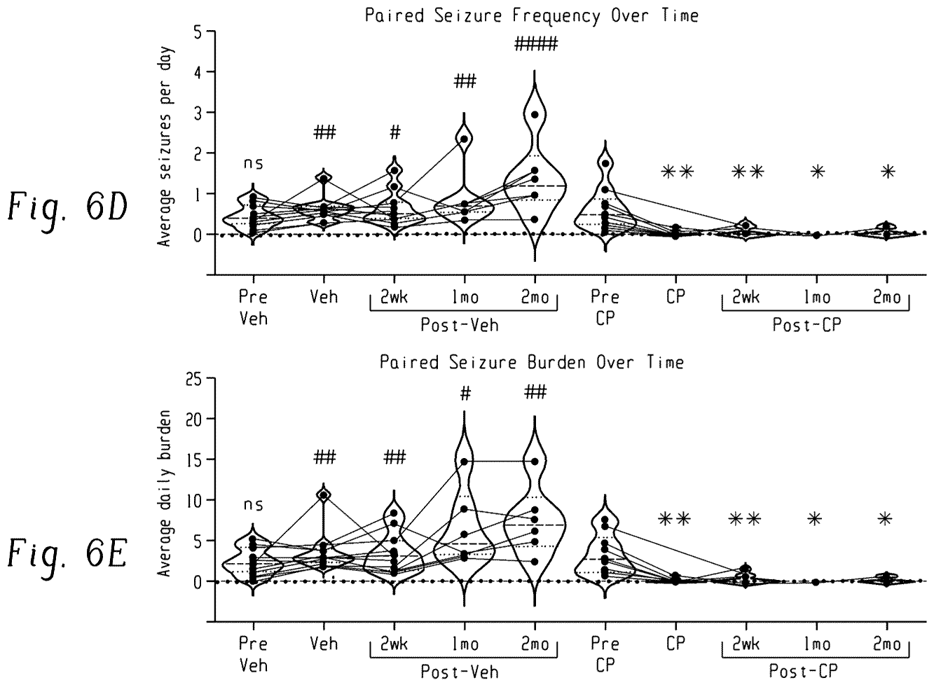

During weeks 11-12 post-SE, drug-treated mice exhibited a 14-fold reduction in seizure frequency and a 22-fold reduction in seizure burden compared to vehicle-treated mice (FIG. 6B-C) (Kolmogorov-Smirnov test; Frequency: D: 1.0, p<10⁻⁴, Burden: D: 1.0, p<10⁻⁴).

### Example 6: CP690550 Reverses Cognitive Comorbidities

It was next determined whether chronically epileptic mice treated with CP690550 showed improvement of epilepsy-associated deficits in spatial memory. Working and short-term spatial memory were tested at three time points: 1-2 weeks prior to SE, during CP690550 treatment at week 12 post-SE, and after drug washout at week 14 post-SE (FIG. 7A).

First, it was determined whether CP690550 could rescue working spatial memory. To do this, spontaneous alternation (SA) was measured in a previously described Y-maze testing paradigm. Mice were placed in the starting arm of the maze facing the end of the arm and allowed to explore freely for 8 minutes. Sequential arm choices during exploration of a three-arm Y-maze are considered an indicator of spatial working memory because the innate curiosity of a healthy mouse promotes exploration of the most novel arm (i.e., the arm least recently visited). Distance traveled during each test was measured to ensure that deficits in SA post-SE were not due to mobility impediments (data not shown). Chronically epileptic mice exhibited a 35% reduction in SA compared to naïve animals (1-way ANOVA; q<10⁻³) (FIG. 7B). In contrast, daily CP690550 treatment rescued spatial memory by 31% (q<0.01), restoring performance to naïve levels (FIG. 7B).

Working memory represents the limited storage of information interfacing with cognition and perception, a process required for the completion of most tasks; this type of memory may degrade rapidly. Short-term memory requires that information collected during an experience be consolidated and accessible shortly thereafter. Thus, in parallel the hypothesis that CP690550 treatment could not only rescue working spatial memory, but also short-term consolidation was tested. Short-term spatial memory was tested with forced alternation (FA) tests in a Y-maze as previously described in the art. Motivated by their innate curiosity, FA tests are contingent on a mouse's ability to recall and apply spatial short-term memory. FA tests involved two 5-minute trials 30 minutes apart: a sample trial and a retrieval trial. In the sample trial, mice were placed in the starting arm and allowed to explore the maze with one arm blocked off. In the retrieval trial, the previously blocked arm was opened, and the mouse was allowed to explore freely for 5 minutes. FA tests were quantified as percentage of time spent in the novel arm.

As with working memory, chronically epileptic mice showed profound deficits in short-term spatial memory, with time spent in the novel arm reduced by 52% at 12 weeks post-SE (1-way ANOVA; q<10⁻³) compared to naïve mice (FIG. 7C). Mice treated daily with CP690550 exhibited a 48% increase (q<0.01) in time spent in the novel arm (FIG. 7C). In contrast, CP690550 administered acutely post-SE has no effect on short-term memory decline (data not shown), highlighting the importance of this second, targetable window.

### Example 7: CP690550 is Disease Modifying

The durability of CP690550's profound effect on seizures and cognition was assessed. Drug was withdrawn and seizure frequency and burden observed from 13-20 weeks post-SE. At the end of week 14 post-SE, SA and FA tests were performed.

CP690550 eliminated seizures in 60% of mice during treatment, and two weeks after drug withdrawal, 60% of mice were still seizure-free (2-way ANOVA; Frequency: 8-fold, q<0.01, Burden: 10-fold, q<0.01) (FIG. 6D-E). Of the mice that were seizure free for the last 3 days of treatment, 75% were seizure free for 2 weeks after drug withdrawal. Seizure suppression persisted to the end of recording. Two months after the last dose of CP690550, a 29-fold reduction in seizure frequency and a 39-fold reduction in seizure burden were observed (2-way ANOVA; Frequency and Burden: q<0.01).

At 2 weeks post-washout, working memory rescued by transient CP690550 treatment persisted with a 37% increase in percent spontaneous alternations compared to vehicle-treated mice (1-way ANOVA; q<10⁻³) (FIG. 7B). Short-term memory as measured by time spent in the novel arm remained indistinguishable from naïve animals two weeks after drug withdrawal, consistent with an ongoing protective effect of CP690550 (FIG. 7C).

Together, these data show that targeting reignition of JAK/STAT signaling in chronic epilepsy represents a novel, powerful disease modifying strategy.

### Example 8: CP690550 vs. Valproic Acid in a Model of Drug-Resistant Epilepsy

Valproic acid is one of the most commonly prescribed anti-epileptic medications. Mice treated with valproic acid take longer to have a seizure as valproic acid is an anti-convulsant with multiple mechanisms of action. Valproic acid can be used as a model for drug-resistant epilepsy.

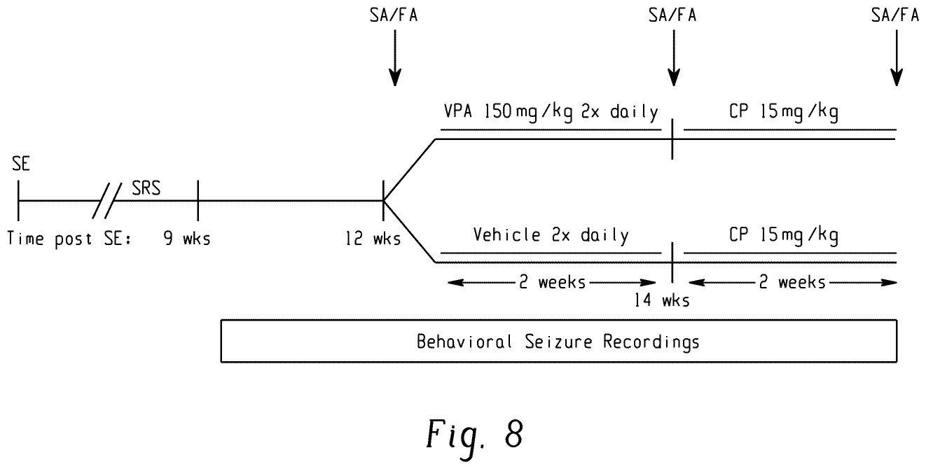

The experimental design is shown in FIG. 8. SE was induced in the mice using a low-dose kainate mouse model as described above. The emergence of spontaneous, recurrent seizures (SRS) after SE was noted. Baseline was established 9 weeks post SE and baseline spontaneous alternation test (SA) and forced alternation test (FA) were determined. At 12 weeks post-SE, either valproic acid (VPA, 150 mg/kg 2 times daily) or vehicle (2 times daily) was administered for 2 weeks. After the 2 weeks, the SA and RA tests were repeated. Then, CP690550 (15 mg/kg) was administered daily for 2 weeks. At 2 weeks after administration of CP690550, the SA and FA tests were again repeated.

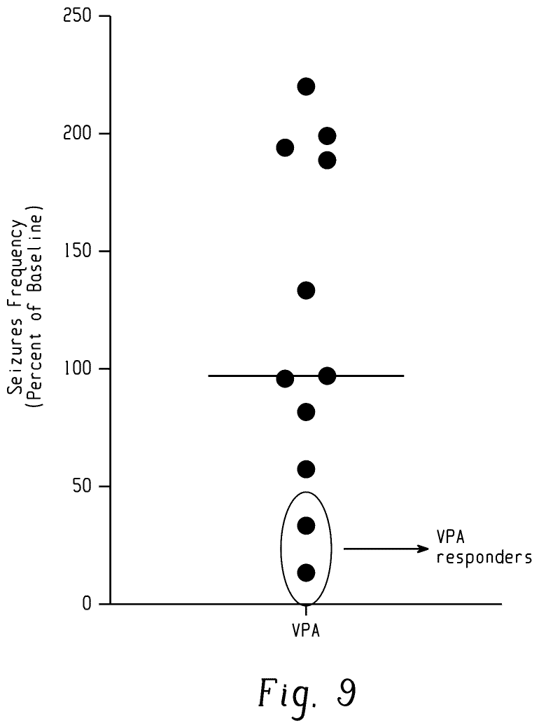

As shown in FIG. 9, VPA failed to suppress seizures, validating drug resistance in the mice. In general, the clinical threshold for effectiveness is a seizure frequency reduction of ≥50%.

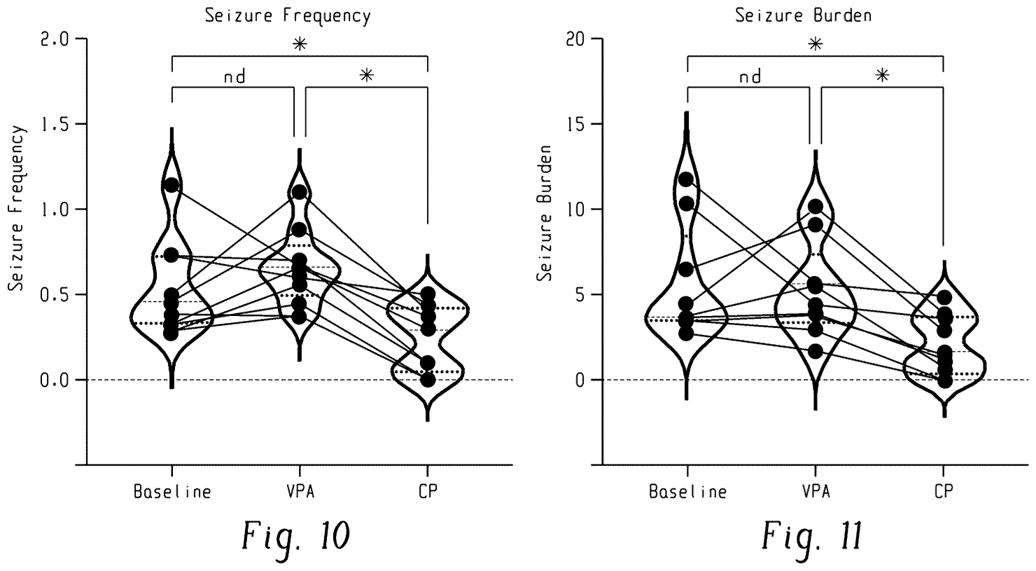

As shown in FIG. 10, for the mice treated with CP690550, the VPA non-responders exhibited a 5-fold reduction in seizure frequency. As shown in FIG. 11, for the mice treated with CP690550, the VPA non-responders exhibited a 3-fold reduction in seizure frequency. Overall, of the VPA non-responder, 67% of mice treated with CP690550 have a 50% or greater seizure reduction compared to VPA.

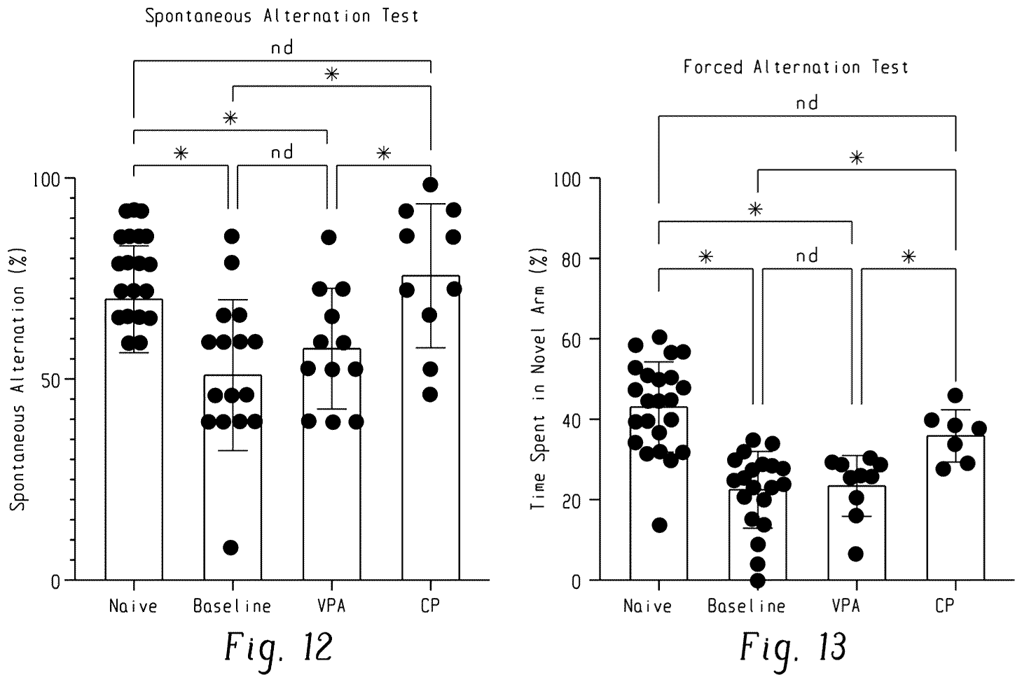

Both epileptic mice and mice treated with VPA have clear memory defects. For the mice treated with CP690550, as determined by spontaneous alternation test (FIG. 12) and forced alternation test (FIG. 13), memory is restored back to baseline levels.

In conclusion, the repeated low dose kainate epilepsy model used here is a model of drug resistant epilepsy. Whereas VPA fails to lower seizure burden and fails to restore spatial memory to these animals, CP690550 both suppresses seizures and restores cognition.

### Example 9: JAK Inhibitor, e.g., Upadacitinib, Treatment Suppresses Electrographic and Behavioral Spontaneous Seizures in Chronically Epileptic Wildtype Mice Across Models

The effect of upadacitinib and other JAK inhibitors described herein on wildtype epileptic mice will be tested. As in Example 5, wildtype mice will be made epileptic via intrahippocampal kainate injections. In a paradigm involving one week of daily vehicle (i.p.) administration followed by an additional week of either vehicle or JAK inhibitor injections under continuous EEG recording, JAK inhibitor treatment during the chronic period will suppress spontaneous seizures. JAK inhibitor-treated mice will also show a significant reduction in seizure frequency and burden over 1 week of recording. JAK inhibitor treatment is expected to robustly reduce both spontaneous seizure frequency and burden in epileptic wildtype mice.

Additionally, while all animals experience seizures during vehicle treatment, a significant fraction of mice are expected to be seizure-free by the third day of JAK inhibitor treatment. Animals treated with vehicle will exhibit seizures throughout the experiment, contrasting with a profound reduction predicted during JAK inhibitor treatment.

### Example 10: JAK Inhibitor, e.g., Upadacitinib, Treatment Reverses Cognitive Comorbidities

It will next be determined whether chronically epileptic mice treated with JAK inhibitor, e.g., upadacitinib, show improvement of epilepsy-associated deficits in spatial memory. Working and short-term spatial memory will be tested at three time points: 1-2 weeks prior to SE, during JAK inhibitor, e.g., upadacitinib, treatment at week 12 post-SE, and after drug washout at week 14 post-SE as in Example 6.

First, it will be determined whether JAK inhibitor, e.g., upadacitinib, can rescue working spatial memory. To do this, spontaneous alternation (SA) will be measured in a previously described Y-maze testing paradigm. Mice are placed in the starting arm of the maze facing the end of the arm and allowed to explore freely for 8 minutes. Sequential arm choices during exploration of a three-arm Y-maze are considered an indicator of spatial working memory because the innate curiosity of a healthy mouse promotes exploration of the most novel arm (i.e., the arm least recently visited). Distance traveled during each test is measured to ensure that deficits in SA post-SE are not due to mobility impediments (data not shown). Chronically epileptic mice may exhibit about a 35% reduction in SA compared to naïve animals. Daily JAK inhibitor, e.g., upadacitinib, treatment is predicted to rescue spatial memory, restoring performance to about naïve levels.

In parallel, the hypothesis that JAK inhibitor treatment could not only rescue working spatial memory, but also short-term consolidation will be tested. Short-term spatial memory will be tested with forced alternation (FA) tests in a Y-maze. FA tests involve two 5-minute trials 30 minutes apart: a sample trial and a retrieval trial. In the sample trial, mice are placed in the starting arm and allowed to explore the maze with one arm blocked off. In the retrieval trial, the previously blocked arm is opened, and the mouse is allowed to explore freely for 5 minutes. FA tests are quantified as percentage of time spent in the novel arm.

As with working memory, chronically epileptic mice are expected to show profound deficits in short-term spatial memory, with time spent in the novel arm reduced compared to naïve mice. Mice treated daily with JAK inhibitor, e.g., Upadacitinib, are predicted to exhibit an increase in time spent in the novel arm. In contrast, JAK inhibitor administered acutely post-SE is predicted to have no effect on short-term memory decline, highlighting the importance of this second, targetable window.

### Example 11: JAK Inhibitor, e.g., Upadacitinib, is Disease Modifying

The durability of JAK inhibitor, e.g., upadacitinib's, profound effect on seizures and cognition will be assessed as in Example 7. Drug will be withdrawn and seizure frequency and burden observed from 13-20 weeks post-SE. At the end of week 14 post-SE, SA and FA tests will be performed.

JAK inhibitor is predicted to eliminate seizures in a substantial fraction of mice during treatment, and two weeks after drug withdrawal, the majority of these mice of mice are predicted to be seizure-free. The majority of seizure-free mice are predicted to be seizure free for 2 weeks after drug withdrawal and to have a reduced seizure frequency and a seizure burden two months after withdrawal.

At 2 weeks post-washout, working memory rescued by transient JAK inhibitor treatment is predicted to persist as evidenced by an increase in percent spontaneous alternations compared to vehicle-treated mice. Short-term memory as measured by time spent in the novel arm is predicted to remain remained indistinguishable from naïve animals two weeks after drug withdrawal, consistent with an ongoing protective effect of JAK inhibitor, e.g., upadacitinib.

### Example 12: JAK Inhibitor, e.g., Upadacitinib, Vs. Valproic Acid in a Model of Drug-Resistant Epilepsy

The experimental design is shown in FIG. 8 and is the same as Example 7. SE will be induced in the mice using a low-dose kainate mouse model as described above. The emergence of spontaneous, recurrent seizures (SRS) after SE will be noted. Baseline will be established 9 weeks post SE and baseline spontaneous alternation test (SA) and forced alternation test (FA) will be determined. At 12 weeks post-SE, either valproic acid (VPA, 150 mg/kg 2 times daily) or vehicle (2 times daily) will be administered for 2 weeks. After the 2 weeks, the SA and RA tests will be repeated. Then, JAK inhibitor, e.g., upadacitinib, will be administered daily for 2 weeks. At 2 weeks after administration of JAK inhibitor, the SA and FA tests will be again repeated.

It is predicted that VPA will fail to suppress seizures, validating drug resistance in the mice. It is further predicted that for the mice treated with JAK inhibitor, the VPA non-responders will exhibit a substantial reduction in seizure frequency. For the mice treated with JAK inhibitor, as determined by spontaneous alternation test and forced alternation test, memory is predicted to be restored back to baseline levels.

### Example 13: Assessing the Degree of Seizure Suppressing Disease Modification by CP690550b and Rinvoq® in the Mouse Systemic Repeated Low Dose Model of Acquired Epilepsy

At 5-7 weeks old, mice will be implanted with radiotelemetry units (HD-X02 from PhysioTel: DSI. 6 week battery life). The units will not be activated at this point. SE will be induced using the repeated low dose kainic acid (KA) paradigm 2 weeks after surgery to allow for recovery. There are no signs of inflammation or STAT activation in the hippocampus 2 weeks post surgery. Kainate injections will be performed at the same time of day (˜10 A.M.), and mice will be returned to home cages before the start of the dark cycle (˜5 P.M.). Mice will be housed with littermates for the duration of the experiment, with males and females separated at weaning. They will be weighed and singly housed in observation chambers for the duration of the injections. Mice will be injected intraperitoneally (i.p.) with 5.0 mg/kg synthetic KA dissolved in 0.9% saline. At twenty-minute intervals, mice will be given 7.5 mg/kg injections of KA up to the third injection and then the dosage will be reduced to 5 mg/kg. Animals will continue to receive 5 mg/kg injections of KA every twenty minutes until each reaches SE. The subsequent KA injection will be skipped if an animal experiences two or more Class V seizures within a single twenty-minute interval. Injections will be resumed for the next round, unless the animal reaches SE. During induction of SE, seizures will be scored using a modified Racine Scale. Behavioral seizure scores will be used to select mice undergoing the same degree of status. Animals will be considered to have reached SE after experiencing at least five Class V or VI seizures within a 90-minute window. KA mice will be observed for 1-2 hours after SE. Animals will be injected with 0.9% saline (s.c.), and soft gel food will be provided in home cages. In the days following injection, animals will be weighed and injected with 0.9% saline (s.c.) if body weight decreases by more than 0.5 g. XO2 units will be activated via magnet and EEG/video monitoring will begin 8 weeks post SE for 1 week to establish baseline seizure burden. Animals that do not exhibit spontaneous seizures during the first recording window will be triaged from the experiment. In previous experiments with FVB mice, >90% of animals go on to develop epilepsy after SE. Animals will be randomly assigned to vehicle or drug group, injected and monitored for 1 week. XO2 units will then be turned off to preserve battery life and turned on for 1 week windows at 2 wks, 1-, 2- and 6-months post SE. At the 1-, 2- and 6-month points, all animals will be moved to assess the impact of drug on comorbid behavioral deficits and endurance of rescue. Seizures will be detected with SeizyML.

Power analysis: n=10 mice per condition to detect 10 fold reduction in seizure frequency given a 10 fold difference in standard deviation from vehicle and drug treated animals, at power=0.9 and alpha=0.1.

Assess the impact of JAK1 inhibitors on behavioral comorbidities associated with epilepsy. The ability of CP690550 or Rinvoq® to protect against, and exert long-term, disease modifying improvements in the cognitive deficits associated with epilepsy will be tested 1-, 3- and 6 months after drug withdrawal measured using the Y-maze, social discrimination, novel object recognition, and Barnes maze and MoSeq.

Assess the degree of disease modification by examining the impact of CP690550 or Rinvoq® on behavioral comorbidities in both models of acquired epilepsy. Chronically epileptic mice will be generated as described above. Only animals that are chronically epileptic (verified by 24/7 video recording) will be used for behavioral analysis. At 0, 1-, 3- and 6 months post-SE, behaviors will be assessed in vehicle or drug treated chronically epileptic mice or controls. Mice will undergo all experimental tests with 24 hours between behavioral assessments in the following order:
Y-maze: Briefly, mice will be placed in the starting arm of the Y-maze and allowed to explore freely for 8 minutes and spontaneous alternation (SA) will be measured as an indication of spatial working memory. Distance traveled will also be measured to control for sickness behavior and/or motor impairments. Short-term memory will be measured using forced alternation (FA) in the Y-maze. Briefly, during the training phase, mice will be placed in the starting arm of the Y-maze with one arm blocked off and allowed to explore for 5 mins. Thirty minutes later, recall testing will be performed by placing mice back into the starting arm with all arms open and allowed to explore for 5 minutes. Mice will normally explore the previously inaccessible/novel arm as a feature of normal exploratory behavior which requires intact short-term memory. (See, FIG. 7A)
Social discrimination: Social discrimination will be used as a test of social recognition memory. The three-chamber social interaction task (3CST) will be performed. Briefly, mice will be placed into a three-chamber apparatus (34″W×11½″D×14″H) in which two wire-mesh cylinders (4″D×4″H) will be placed in each of the left and right chambers. The mice will be individually placed into the center chamber of the apparatus and allowed to habituate and explore the environment for 5 mins. After the 5 min habituation session, social interaction will be measured by placing an unfamiliar conspecific mouse into one of the cylinders. The position of the mouse will be alternated and counterbalanced between sessions to avoid a place preference bias. During the social interaction test session, the amount of time spent in the interaction zone (6″D) of the cylinder containing the conspecific mouse will be measured during a 10 min period to assess motivation for social interaction. A social interaction ratio will be calculated as the ratio of time spent in the interaction zone in the target session relative to the empty cylinder. The social discrimination session will be performed after a 24 hr retention interval and will involve placing the previous conspecific mouse into one chamber and a novel, unfamiliar conspecific into the other chamber. Mice will be placed into the center chamber of the apparatus and allowed to explore for 10 mins. The amount of time spent in the interaction zone with the familiar and unfamiliar conspecific will be measured. All sessions will be evaluated for subject preference ratios by an investigator blind to the experimental group. The location of the familiar and unfamiliar mice (left versus right end of the chamber) will be counterbalanced across experimental groups to control for nonspecific location preference effects.
Novel object recognition: Novel object recognition will be used to assess memory in mice as previously. Mice will be acclimated to the novel object recognition chamber (6″W×10″D×5.5″H) for 10 min daily for three days before testing. On the first testing day, each mouse will be individually placed into the center of the chamber with two identical objects fixed to the floor at opposite ends of the chamber. Mice will be allowed to explore the chambers for 5 min and this familiarization trial will be conducted three times, with a 15 min interval between trials. After a 3-hour retention interval, each mouse will be individually placed into the chamber with one familiar object and one novel object of similar sizes. The mice will be allowed to examine the objects for a 10 min testing period which will be videotaped. All sessions will be evaluated by an investigator blinded to experimental condition for object preference ratios. The location of the novel object (left versus right end of the chamber) will be counterbalanced across experimental groups to control for nonspecific location preference effects.
Barnes maze: The Barnes maze will be used to measure spatial learning and memory in mice. Mice will undergo habituation, training, and testing phases using a Barnes maze which consists of a large circular platform (100 cm diameter) with 20 equally spaced holes along the perimeter (each 5 cm diameter) with a small, black escape box placed under one of the holes. Visual cues with different shapes and colors will be placed on the walls surrounding the apparatus. For the habituation phase on Day 1, mice will be placed into the escape box for 1 min. Mice will then be individually placed into the center of the apparatus and allowed to explore until it locates the escape box, or 5 mins has elapsed. Acquisition training sessions will be performed over a 3 min period, twice per day with a minimum of 1 hour between trials on Days 1-10 which involves placing the mice into the center of the apparatus with the escape box in a fixed position. Three days following training, the mice will be tested in a 1 min probe trial in which the mouse is placed into the center of the Barnes maze with no escape chamber. The latency to locate and enter the escape box, path length, and strategies will serve as outcome measures. The behavioral comorbidities will be correlated with seizure burden using a linear regression analysis.

The proposed behavioral approaches listed above are well validated approaches for interrogating cognitive impairments associated with epilepsy related to learning and memory deficits. While this is a powerful approach, appropriate for evaluating cognitive impairments associated with epilepsy, and the ability of CP690550 or Rinvoq to mitigate those impairments, these approaches focus on specific, well-but pre-defined outcome measures. In an effort to obtain a more holistic and unbiased view of the impact of JAK1 inhibitors on behavioral comorbidities, Motion Sequencing (MoSeq) will also be employed, a machine learning-assisted 3D video analysis approach, to assess “hidden” behavioral phenotypes in mice associated with epilepsy. This approach has been used to characterize the underlying structure of spontaneous behavior in mice as well as screen antiseizure treatments. This approach will be used to characterize behavioral comorbidities in mice with unprecedented resolution as well as determine whether CP690550 exerts disease modifying effects which can be measured by MoSeq. Specifically, MoSeq will be used to deconstruct behavior at sub-second timescales into stereotyped, recurrent occurring modules (sub-second behavioral “syllables”) that are arranged according to specific transition rules (“grammar”) in both models generated as described above. This evolution of behavioral comorbidities can be evaluated over time at 2 weeks, 1, 3 and 6 months post-SE.

CP690550 acts centrally and targets brain JAK1/3. Rinvoq® has virtually identical albumin binding profiles to CP690550 and a significantly longer T12. It is predicted that Rinvoq® will be potently seizure suppressing and disease modifying.

It is predicted that CP690550 and the more specific JAK 1 inhibitor Rinvoq®, will exert long-term improvements in working memory in chronically epileptic mice, lasting 6 months following treatment. Typically, mice prefer to interact with an unfamiliar mouse which requires memory of previous social interaction exposure. It is predicted that chronically epileptic mice will exhibit deficits in social discrimination, in novel object recognition, Barnes maze (longer latency, longer path length, and differing/random strategy) compared to controls and that CP690550 treatment will exert long-term improvements in chronically epileptic mice, lasting 6 months following treatment. It is predicted that chronically epileptic mice will exhibit altered behaviors, evidenced by changes in the “syllables” and “grammar” in the MoSeq paradigm expressed by epileptic compared to controls. Further, it is predicted that CP690550/Rinvoq® treatment will improve behavioral outcomes in epileptic mice, restoring their “syllables” and “grammar” to those exhibited by controls. There may be an impact of repeated testing in the same cohort of animals-especially for tasks like the Barnes maze where mice may become habituated to the test. If of habituation is observed (by consistent improvement in performance by naïve mice), cohort batches of mice will be used for each time point. A major challenge is being able to dissociate the impact of CP690500 or Rinvoq® on seizure activity and the impact on behavioral comorbities. However, the goal is to demonstrate the disease modifying effects of these compounds and improvement in either or both of these outcome measures would demonstrate potential benefit of these treatments. Because CP690550 and presumably also Rinvoq® can be administered transiently in the chronic period to give enduring protection would largely mitigate the risk of systemic immunological effects associated with long-term use of JAK inhibitors.

### DISCUSSION

It has been shown herein that that rapid induction of JAK/STAT3 signaling with SE is quenched by the chromatin modifier EZH2, but reignites in the chronic period. Targeting this resurgence with CP690550 (tofacitinib), an FDA-approved, orally available drug, potently suppresses seizures and reverses cognitive comorbidities in an enduring manner. The JAK inhibitor CP690550 is a novel, disease-modifying therapeutic agent for seizure suppression and cognitive restoration across epilepsy models. It is predicted that other JAK1 and 1/3 inhibitors such as Upadacitinib will provide similar results.

After status epilepticus, EZH2 protein is induced in hippocampal pyramidal and dentate granule neurons across models of epilepsy. Previously, it was reported that treatment of mice with the SAM-competitive EZH2 inhibitor UNC-1999 exacerbated spontaneous seizures, and as shown herein deletion of EZH2 in mature neurons has the same effect. Induction of Polycomb group (PcG) proteins has been studied in association with other brain insults. For example, sublethal ischemic insults lead to induction of Polycomb in mouse cortical neurons. Further, sevoflurane (a volatile anesthetic that exerts positive allosteric modulation on GABA_{A} receptors and antagonizes NMDA receptors) mediates the protective induction of EZH2 and H3K27Me3 to attenuate hypoxic-ischemic brain injury, reducing hippocampal autophagy and improving post-injury behavioral assessments in rats. These improvements are reversed when rats treated with sevoflurane also receive a dose of the EZH2 inhibitor GSK126. These studies, along with the findings herein, point to a potentially general protective role for EZH2 in brain insults.

EZH2 deletion in neurons results in large transcriptomic changes after SE (FIG. 2). The mouse Black gene cluster houses genes whose expression are induced after SE in EZH2nWT mice and hyper-induced in EZH2nHom mice after SE. This pattern suggests that EZH2 is responsible for tempering expression of genes in this cluster because its deletion results in their unfettered activation. The mouse Black cluster ontological analysis produces terms associated with innate immunity, neuroinflammation, and the JAK/STAT pathway. Network analysis places STAT3 in the center of the mouse Black GRN.

Increased activation of STAT3 at Tyr705 as a result of EZH2 inactivation has also been observed in a transformed murine cell model of early T cell precursor acute lymphoblastic leukemia; EZH2 inactivation leads to enrichment of documented STAT3 target genes and exaggerated pSTAT3 induction with IL6 stimulation. STAT-upstream receptors and kinases are also regulated by EZH2. In prostate cancer EZH2 represses IFNGR1 to dampen JAK2/STAT1 signaling, and its depletion restores expression of IFN target genes and pSTAT1.

While activation of the JAK/STAT pathway in non-neuronal cells post-SE is well-characterized, the role of STAT3 in neurogenic inflammation (data not shown) post-SE is not clear. Cytokines released from glial cells following seizures increase neuronal excitability, and type I IFNs increase excitability in neocortical pyramidal neurons via PKC. It has been reported that seizure-induced activation of the JAK/STAT pathway reduces the number of gamma-aminobutyric acid (GABA) type A alpha1 receptors expressed in neurons following seizures, influencing GABA (A) R-dependent inhibition.

Pharmacological inhibition of STAT3 prior to or during SE blunts, but does not prevent onset of spontaneous seizures. Though mechanistically enlightening, this approach has little clinical utility. Attempts to genetically ablate of the STAT3 SH2 domain plateaued disease progression but did not reduce seizure burden, and the impact on comorbidities was unclear due to profound differences in the naive wildtypes and STAT3-SH2 knockout mice. Inhibiting the first wave of JAK/STAT signaling has no effect on spontaneous seizures and epilepsy-associated memory decline (data not shown). Targeting the second wave of STAT3 activation in chronic epilepsy with the JAK inhibitor CP690550 results in an enduring, disease-modifying suppression of spontaneous seizures and the restoration of spatial memory (FIG. 6-7).

Analysis of human TLE samples identifies a cluster of genes strikingly similar to the STAT3-driven mouse Black cluster; the human Brown cluster is a group of genes associated with innate immunity and neuroinflammation (FIG. 3). Like the mouse Black cluster, the most influential driver of gene expression is STAT3. The robust overlap between clusters of genes, one from frankly epileptic human samples and another from rodents in an early epileptogenic period, suggests a couple of insights. Firstly, STAT3-mediated epileptogenic mechanisms identified in mice are clinically relevant to the human population. Secondly, STAT3 drives gene changes both acutely after insult and in chronic epilepsy; this is supported by the identification of STAT3 as a driver of gene expression, its transient activation acutely post-SE, and its reactivation in chronic rodent epilepsy (FIG. 4).

In some cases, the struggle to translate novel therapies from preclinical rodent models to humans may stem from unreconciled differences between mechanisms that are active acutely in rodents and those which are active in chronically epileptic human patients. In other words, treatments administered acutely to thwart disease in rodents may target mechanisms that are not active in chronic epilepsy. However, other pathways hitherto described as activating acutely post-SE and then abating ought to be reexamined and screened for reignition in chronic epilepsy. Herein, provided is the first proof-of-principle that a mechanism thought to be malleable only in a brief, critical window shortly following SE is in fact relevant across preclinical rodent models, active in human disease, and targetable for potent disease modification in chronic epilepsy.

CP690550 profoundly suppresses seizures in chronically epileptic wildtype mice across models of epilepsy as measured by two different methods of seizure detection. Following cessation of CP690550 treatment robust disease modification is observed for at least two months. The finding that CP690550 can be administered transiently in the chronic period to give lasting seizure protection would largely mitigate the risk of systemic immunological effects associated with long-term use of JAK inhibitors, as in rheumatoid arthritis (RA). RA patients may require up to 6 months of CP690550 treatment before improvements begin to manifest. In contrast, observed herein is profound disease modification after two weeks of daily CP690550 treatment. Given the rapid suppression of seizures, future studies will seek to shorten the course of treatment.

The disease modifying properties of agents like dimethyl fumarate (DMF) and sodium selenate have been reported recently. Like CP690550, DMF is FDA-approved, and its administration in chronic epilepsy suppresses seizures during treatment and after drug washout. Whereas DMF was able to enduringly suppress seizures when administered to chronically epileptic rats, DMF has not been shown to rescue cognition with chronic administration, and administration at the time of insult, as previously highlighted, has little clinical utility.

Cognitive decline is not only a challenge with current epilepsy therapies, but also a leading comorbidity of epilepsy itself. Despite recent advances, there is a continued, critical need for disease modifying agents that address comorbid behavioral and cognitive decline. The mechanism(s) linking epilepsy to comorbidities is still unclear, but inflammation is a commonality across epilepsy and co-occurring conditions such as cognitive dysfunction, depression, anxiety, and schizophrenia. Indeed, others have shown that suppression of key neuroinflammatory mediators such as COX2-associated pathways and Complement 3-dependent synaptic remodeling rescues aspects of cognitive function in preclinical rodent models of acquired epilepsy. It is still unclear whether seizures are the primary cause of neuroinflammation and comorbid cognitive decline observed in people with epilepsy, or if seizures and cognitive decline share a common cause in neuroinflammation.

In addition to profound seizure suppression, CP690550 treatment during the chronic period enduringly alleviated epilepsy-associated deficits in spatial memory. For both working and short-term memory, treated animals are indistinguishable from naïve animals both during treatment and two weeks after drug washout. For FA tests performed post-washout, time spent in the novel arm (FIG. 7C) did not differ robustly between CP690550- and vehicle-treated mice. This is most likely due to a large variance in the post-vehicle cohort, rather than a reduced mean of the post-CP690550 cohort. The role of inflammation in epilepsy-related cognitive decline has been explored. An EP2 antagonist TG11-77 is an investigational new drug that reduces microgliosis and improves spatial working memory in a pilocarpine model of epilepsy. CP690550 is the first pharmacological agent that may be administered transiently several weeks after the onset of spontaneous seizures to rescue spatial memory for weeks after drug withdrawal, and provide enduring seizure protection for at least two months after drug withdrawal.

Described herein is an open discovery, omics-based approach involving multiple animal models and laboratories to identify drivers of disease, paired with a concerted effort to confirm relevance to human disease. With this approach EZH2 was identified as a protective negative regulator of the JAK/STAT pathway in epilepsy. The current data is consistent with the hypothesis that CP690550 mitigates spontaneous seizures in chronic epilepsy and is disease-modifying. The cross-model, systems-based approach by which we identified JAK/STAT signaling as a target of EZH2 and selected CP690550 suggests this drug may be useful across multiple human epilepsies upon clinical translation.

Characterization of an endogenous, EZH2-mediated, protective response across rodent models of epilepsy has highlighted a biphasic activation of JAK/STAT3 signaling: an early induction and abatement after insult followed by reactivation with chronic, spontaneous seizures. Transiently targeting STAT3 reignition in chronic epilepsy with the FDA-approved drug CP690550 is potently seizure suppressing, memory restoring, and disease modifying.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of treating a patient with epilepsy or suspected of having epilepsy, comprising administering a Janus Kinase inhibitor to the patient.

2. The method of claim 1, wherein the patient is being treated for an autoimmune disease or atopic dermatitis, and the patient has not previously been administered a Janus Kinase inhibitor.

3. The method of claim 1, wherein the patient is not being treated for an autoimmune disease or atopic dermatitis and/or has not been diagnosed with an autoimmune disease or atopic dermatitis.

4. The method of claim 1, wherein the patient is a human patient.

5. The method of claim 1, wherein the patient is a dog.

6. The method of claim 1, wherein the Janus Kinase inhibitor is administered for a first period of time, and is then subsequently withdrawn for a second period of time, wherein seizures are suppressed in the patient during the second period of time.

7. The method of claim 6, wherein the patient is a human patient, and the first period of time is 1 week to 12 weeks and the second period of time is 1 week to the lifetime of the patient.

8. The method of claim 6, wherein the patient is a human patient, and the first period of time is 1 week to 4 weeks.

9. The method of claim 6, wherein the patient is a human patient, and the first period of time is 1 week to 2 weeks.

10. The method of claim 6, wherein the patient is a human patient, and the second period of time is 1 month to 6 months.

11. The method of claim 6, wherein the patient is a human patient, and the second period of time is 1 to 4 weeks.

12. The method of claim 6, further comprising, during the second time period, assessing behavioral and/or cognitive ability in the patient.

13. The method of claim 12, wherein behavioral and/or cognitive abilities comprise spatial memory, cognitive dysfunction, depression, anxiety, schizophrenia, or a combination thereof.

14. The method of claim 1, wherein the Janus Kinase inhibitor is tofacitinib.

15. The method of claim 14, wherein the tofacitinib is administered orally daily.

16. The method of claim 14, wherein the daily dosage of tofacitinib is 5 to 22 mg/day.

17. The method of claim 1, wherein the Janus Kinase inhibitor is upadacitinib, abrocitinib, baricitinib, filgotinib, ritlecitinib, peficitinib, decernotinib, or oclacitinib.

18. The method of claim 1, wherein the Janus Kinase inhibitor is administered after an episode of status epilepticus.

19. The method of claim 1, wherein the patient has genetic epilepsy.

20. The method of claim 1, wherein the patient has acquired epilepsy.

21. The method of claim 1, wherein the patient has drug-resistant epilepsy.

22. The method of claim 1, wherein administering the Janus Kinase inhibitor to the patient reduces seizure burden, reduces seizure frequency, restores memory as evidenced by spontaneous alternations test, and/or restores memory as evidenced by forced alternation test.

23. The method of claim 1, further comprising administering an anti-seizure medication to the patient.

24. The method of claim 23, wherein the anti-seizure medication comprises brivracetam, cannabidiol, carbamazepine, cenobamate, clobazam, clonazepam, divalproex, phenytoin, eslicarbazepine, ethosuximide, felbmate, fenfluramine, gabapentin, ganaxolone, lacosamide, lamogitrine, levitracetam, methsuximide, midazolam, oxcarbazepine, perampanel, phenobarbitone, potiga, pregabalin, primidone, rufinamide, tiagabine, topiramate, vigabatrin, zonisamide, or a combination thereof.
